(19) United States
(12) Patent Application Publication
(10) Pub. No.: US 2026/0144719 A1
(43) Pub. Date: May 28, 2026

(54) DENTAL POWDER AND METHOD OF DENTAL HEALTH FOR ANIMALS

(71) Applicant: PetHonesty, LLC, Austin, TX (US)

(72) Inventors: Gregory Allen Reinhart, South Lebanon, OH (US); John Erick Szramiak, Austin, TX (US); Ryan Allan Frank, Fairview, TN (US)

(21) Appl. No.: 19/325,674

(22) Filed: Sep. 11, 2025

Related U.S. Application Data
(60) Provisional application No. 63/724,784, filed on Nov. 25, 2024.

(51) Int. Cl. A61K 8/02; A61K 8/9711; A61K 8/99; A61Q 11/00

(52) U.S. Cl. CPC A61K 8/022; A61K 8/9711; A61K 8/99; A61Q 11/00; A61K 2800/92

(57) ABSTRACT

Provided herein are methods and compositions for a dental powder and dental health for animals.

### CROSS-REFERENCE TO RELATED APPLICATIONS

The present application claims priority to U.S. application Ser. No. 63/724,784, filed Nov. 25, 2024, herein incorporated by reference in its entirety.

### BACKGROUND

The invention generally relates to dental powder and oral health of pets.

Oral health is an important issue for domesticated animals such as dogs and cats. The diet of such animals, e.g., canned and dried foods, may not include natural teeth cleaning agents that would be found in a traditional diet. As a result, the American Veterinary Dental College estimates that 80% of dogs and 70% of cats over the age of three develop oral disease, i.e., an infection of the teeth, gums, and mouth, which can lead to serious health issues. More specifically, oral disease may start off with bad breath and discolored teeth but can progress to cessation of eating dried food or chewing toys due to discomfort and pain. If untreated, it can lead to tooth loss and, in some instances, heart disease as bacteria in the mouth pass into the bloodstream and accumulate in pets' arteries. Conventional oral care prevention typically involves expensive oral examination, scaling and polishing of teeth to remove tartar and plaque. These procedures require general anesthesia and pre-anesthesia blood work.

In addition, prescription diet animal dry food may reduce plaque, stain & tartar buildup; however, these prescription diet dry foods often lack optimal nutrition and include less protein and other nutrients necessary for dogs and animals to thrive. Dogs and animals are often undernourished when eating prescription diet dry food. So in order to maintain nutritional levels for dogs and animals and maintain dental health, a dental powder application to proper animal feed or food product may provide the necessary oral and dental health of animals when eating a diet with sufficient protein and vitamins.

The present invention attempts to solve these problems as well as others.

### SUMMARY OF THE INVENTION

Provided herein are methods and compositions for a dental powder and dental health for animals.

The methods and compositions are set forth in part in the description which follows, and in part will be obvious from the description, or can be learned by practice of the methods and compositions. The advantages of the methods and compositions will be realized and attained by means of the elements and combinations particularly pointed out in the appended claims. It is to be understood that both the foregoing general description and the following detailed description are exemplary and explanatory only and are not restrictive of the methods and compositions, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53 (c) EPC and Rule 28 (b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### BRIEF DESCRIPTION OF THE DRAWINGS

In the accompanying figures, like elements are identified by like reference numerals among the several preferred embodiments of the present invention.

FIG. 1A is a perspective view of the dental powder, according to one embodiment.

FIG. 1B is a graph of the % of pet parents who saw improvement in the dog's breath odor, tooth whiteness, visible plaque and tartar, gum health and overall dental health by administering the dental powder to their pet from week 1 to week 8.

FIG. 2 is a graph of the Intake Ratio Chart for the individual Intake Ratios for each of the 60 dogs are sorted in ascending order for Chow+PH Dental Powder.

FIG. 3 is a graph of the average daily consumption of each diet for 5 days.

FIG. 4 is a graph of the diet of first choice preference scored daily for each dog for 5 days.

FIG. 5 is a graph of the preference scoring by intake ratio.

FIG. 6 is a graph of the day 1 food consumption and first choice data for each dog.

FIG. 7 is a graph of the day 2 food consumption and first choice data for each dog.

FIG. 8 is a graph of the day 3 food consumption and first choice data for each dog.

FIG. 9 is a graph of the day 4 food consumption and first choice data for each dog.

FIG. 10 is a graph of the day 5 food consumption and first choice data for each dog.

FIG. 11 is a graph of the total food consumption and first choice data for each dog.

FIG. 12 is a graph of the individual Intake Ratios for each of the 20 dogs are sorted in ascending order for Chow+PH Dental and Chow+PlaqueOff in Example 2.

FIG. 13 is a graph showing the average daily consumption of each diet where the consumption of each diet is compared using a separate paired t-Test within each day (20 dogs by 2 diets) to assess significance in consumption difference for that day of Chow+PH Dental and Chow+PlaqueOff.

FIG. 14 is a graph showing the diet of First Choice preference is scored daily for each dog and the counts of Chow+PH Dental and Chow+PlaqueOff.

FIG. 15 is a graph showing using the 2:1 consumption criteria in the Intake Preference chart to the right, 10 dogs prefer Chow+PH Dental, no dogs prefer Chow+PlaqueOff and the other 10 dogs show no Preference.

FIG. 16 is a graph showing the Day 1 food consumption and first choice data of Chow+PH Dental and Chow+PlaqueOff.

FIG. 17 is a graph showing the Day 2 food consumption and first choice data of Chow+PH Dental and Chow+PlaqueOff.

FIG. 18 is a graph showing the total food consumption and first choice data of Chow+PH Dental and Chow+PlaqueOff.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### DESCRIPTION OF EMBODIMENTS

Generally speaking, the dental powder composition comprises kelp, sodium hexametaphosphate, a botanical blend, and a postbiotic. The dental powder composition is applied to a food product for a pet or animal and increases dental health by insuring adherence and consumption of the dental powder composition for dental health, oral health, and teeth cleaner. In other embodiments, the dental powder composition comprises inactive ingredients including Bentonite, Brown Rice, Hydrolyzed Yeast, Mixed Tocopherols, (Preservative), Natural Mint Flavor, Natural Pork Flavor.

Disclosed herein is a method for treating, reducing, or ameliorating one or more oral conditions in a user, human, or animal subject in need thereof with the dental powder compositions including the one or more kelp, sodium hexametaphosphate, a botanical blend, and a postbiotic and/or parts thereof. As used herein “animal subject” may include higher order non-human mammals such as canines, felines, pigs, and horses. The one or more oral conditions may include any oral conditions treatable with any one or more therapeutic benefits of the dental powder composition and/or parts thereof. As discussed above, the one or more therapeutic benefits may be or include, but are not limited to, breath odor, tooth whiteness, visible plaque and tartar, gum health and overall dental health, or the like, or any combination thereof. The method may include contacting the dental powder composition with surfaces of the oral cavity during an eating period.

For example, the method may include chewing the dental powder composition in the oral cavity to contact the dental powder composition with surfaces of the oral cavity. The method may include contacting the dental powder composition with surfaces of the oral cavity at least once per day, at least twice per day, at least three times per week, or more.

### Composition

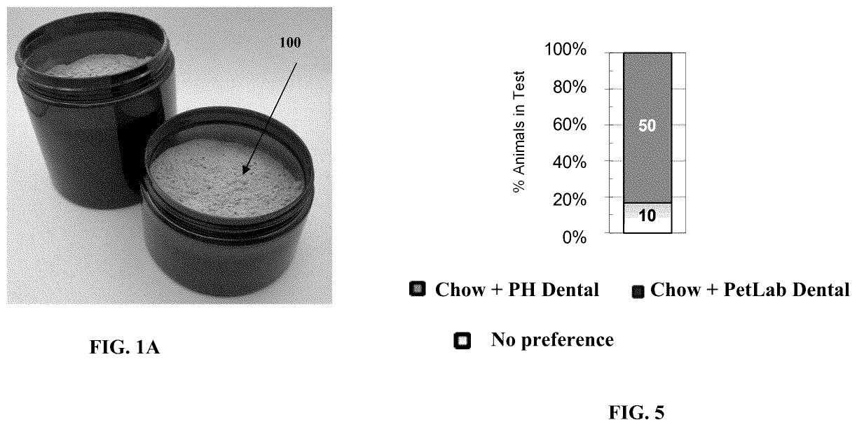

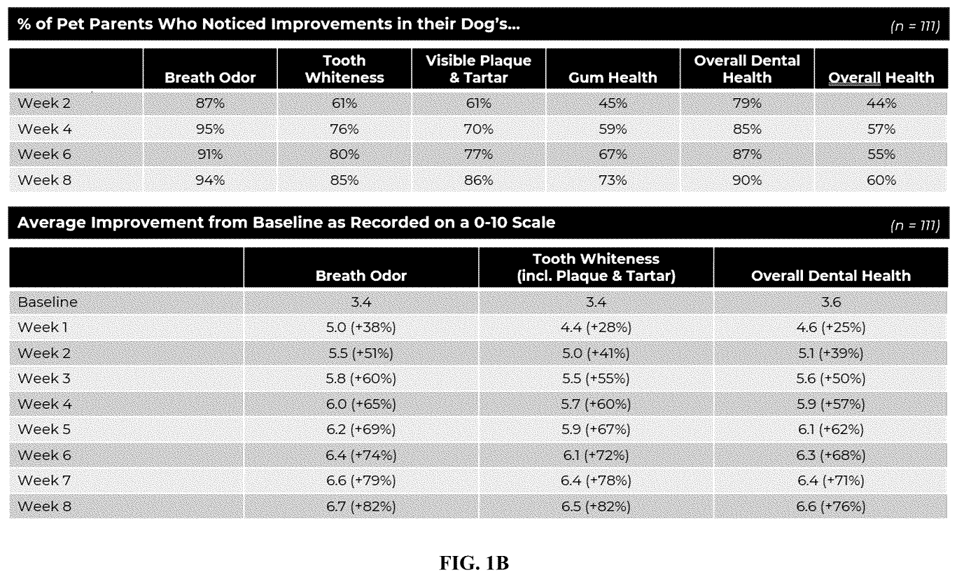

Compositions disclosed herein may be or include a dental powder product or a dental powder composition thereof, as shown in FIG. 1A. For example, the compositions disclosed herein may be an oral care composition, including the dental powder composition, or the dental powder composition thereof. In at least one implementation, the dental powder composition may include an orally acceptable vehicle or carrier and one or more of seaweed, sodium hexametaphosphate, a botanical blend, and a postbiotic and/or parts thereof. For example, the dental powder composition may include an orally acceptable vehicle and one or more sea vegetable, anti-tartar agent, a botanical blend, and a probiotic. In another example, the dental powder composition may include an orally acceptable vehicle and one or more parts of a kelp, sodium hexametaphosphate, a botanical blend, and a prebiotic. In yet another example, the dental powder composition may include an orally acceptable vehicle, and one or more parts of a sea vegetable, anti-tartar agents, a botanical blend, and a postbiotic. In at least one implementation, the dental powder compositions disclosed herein are intended to be part of a diet and may be ingested. For example, any therapeutic benefits disclosed herein may be provided by contact with surfaces of the oral cavity and with ingestion.

The one or more seaweed, sodium hexametaphosphate, a botanical blend, a postbiotic and/or parts thereof may be mixed, combined, disposed, suspended, or otherwise contacted with the orally acceptable vehicle. In at least one implementation, the seaweed, sodium hexametaphosphate, a botanical blend, and a postbiotic and/or parts thereof may generally be evenly distributed throughout the orally acceptable vehicle. For example, the one or more seaweed, sodium hexametaphosphate, a botanical blend, and a postbiotic and/or parts thereof may be suspended and substantially evenly distributed throughout the orally acceptable vehicle. In another implementation, the one or more seaweed, sodium hexametaphosphate, a botanical blend, and a postbiotic and/or parts thereof may be disposed in the orally acceptable vehicle and the one kelp, sodium hexametaphosphate, a botanical blend, and a postbiotic and/or parts thereof may be generally concentrated in a portion of the orally acceptable vehicle. For example, the orally acceptable vehicle may be disposed in a container or vessel, and the one or more seaweed, sodium hexametaphosphate, a botanical blend, and a postbiotic and/or parts thereof may be disposed in the orally acceptable vehicle and settled at a bottom portion of the container (e.g., settling via density). In yet another implementation, at least a portion of the one or more seaweed, sodium hexametaphosphate, a botanical blend, and a postbiotic and/or parts thereof may be generally distributed throughout the orally acceptable powder and at least another portion may be generally concentrated in the orally acceptable powder.

Lactobacillus plantarum or Postbiotic, Probiotic, Prebiotic

Lactobacillus plantarum (L. plantarum) is a type of probiotic or postbiotic and L. plantarum can help break down food, absorb nutrients, and fight off “bad” organisms that might cause oral diseases. L. plantarum help with oral health by inhibiting the growth of harmful bacteria and reducing the risk of cavities.

L. plantarum inhibits oral pathogens and L. plantarum can reduce the number of bacteria and change the structure of biofilms caused by S. mutans, a major bacterium that causes dental caries. It can also combat other oral pathogens, such as Aggregatibacter actinomycetemcomitans and Prevotella intermedia. L. plantarum has a low adhesion ratio to hard tissue, which means it's less likely to cause cavities. L. plantarum can survive in acidic environments and colonize the oral cavity.

In another embodiment, the postbiotic, prebiotic, or probiotic is selected from one or more of the genera selected from: Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus and Weissella. In an embodiment, the lactic acid bacteria is selected from one or more or all of: Lactobacillus plantarum, Leuconostoc mesenteroides, Lactobacillus rhamnosus, Lactobacillus pentosus, Lactobacillus brevis, Lactococus lactis, Lactobacillus acidophilus, Lactobacillus brevis, Lactobacillus casei, Lactobacillus delbrueckii, Lactobacillus fermenturn, Lactobacillus gasseri, Lactobacillus johnsonii, Lactobacillus lactis, Lactobacillus paracasei, Lactobacillus reuteri, Pediococcus pentosaceus and Pedicoccus acidilacti.

The amount of the probiotic, prebiotic, or postbiotic present in the dental powder product or the dental powder composition thereof may be from about 0.001 weight percent to about 3 weight percent, based on a dried, rehydrated, or fresh weight of the probiotic, prebiotic, and/or postbiotic relative to a total weight of the dental powder product or the dental powder composition thereof. For example, the amount of the one or more probiotic, prebiotic, and/or postbiotic present in the dental powder product or the dental powder composition thereof may be from about 0.001 weight percent, 0.005 weight percent, about 0.01 weight percent, about 0.05 weight percent, about 0.1 weight percent, about 0.5 weight percent, about 1 weight percent, about 5 weight percent, about 1.0 weight percent, or about 1.5 weight percent to about 2.0 weight percent, about 2.5 weight percent, or about 3.0 weight percent, based on a dried, rehydrated, or fresh weight of the one or more probiotic, prebiotic, postbiotic and/or parts thereof relative to a total weight of the dental powder product or the dental powder composition thereof.

### Kelp or Seaweed

Kelp or seaweed can treat and maintain dental health and oral health because kelp contains natural compounds that reduce plaque and tartar and protect against tooth decay. Kelp contains an enzyme called Bacillus licheniformis that breaks down plaque and tartar precursors. This enzyme can also remove large amounts of plaque from teeth. Seaweed has antibacterial properties that can remove harmful bacteria while leaving beneficial probiotic bacteria in place.

Kelp or sea vegetables (also known as algae) include green, brown, red, and blue-green algae. Sea vegetables include kelp (laminaria), which is a brown marine plant that contains vitamins A, B, E, D and K, is a key source of vitamin C, and is rich in minerals. Kelp proteins are comparable in quality to animal proteins. Kelp also contains sodium alginate (algin), an element that helps remove radioactive particles and heavy metals from the body. In one embodiment, the kelp used is ascophyllum nodosum.

Derivatives of algae include chlorophyll, carrageenan, carotenoids (e.g., fucoxanthin), fucoidan (a carbohydrate from brown algae), DNA, and RNA. Algin, carrageenan, and agar are kelp gels that rejuvenate gastrointestinal health and aid digestion. Kelp helps purify blood, relieves arthritis stiffness, and promotes adrenal, pituitary, and thyroid health. Kelp contains iodine, which can be used to normalize thyroid-related disorders.

Kombu (laminaria digitata, setchelli, horsetail kelp) is another sea vegetable with great nutritional healing value. It is a decongestant for excess mucous and helps normalize blood pressure. Kombu has abundant iodine, carotenes, B, C, D and E vitamins, minerals like calcium, magnesium, potassium, silica, iron and zinc, and the powerful skin healing nutrient germanium. Kombu is meaty, high-protein seaweed. It is higher in natural mineral salts than most other seaweeds.

Hijiki is a sea vegetable that is a mineral-rich, high-fiber seaweed, with 20% protein, vitamin A, carotenes and calcium.

Another sea vegetable, nori (porphyra, laver), is a red sea plant with a sweet, meaty taste when dried. Nori contains nearly 50% balanced, assimilable protein, which is more than any other sea plant. Nori is rich in all the carotenes, calcium, iodine, iron, and phosphorus.

Another sea vegetable, arame (Eisenia bycyclis), is one of the ocean's richest sources of iodine. Herbalists use arame to help reduce breast and uterine fibroids, adhesions, and to normalize menopausal symptoms through arame's fat-soluble vitamins and phytohormones. Arame promotes soft, wrinkle-free skin, enhances glossy hair and prevents its loss. Sea palm (Postelsia Palmaeformis), which is also known as American arame, grows only on the west coast of North America.

Another sea vegetable, bladderwrack, is rich in vitamin K and potassium and is an excellent adrenal stimulant. Another sea vegetable, wakame (alaria, undaria), is a high-protein, high-calcium seaweed with carotenes, iron, and vitamin C. Another sea vegetable, dulse (palmaria palmata), is a red sea plant that is rich in iron, protein, and vitamin A. Dulse has 300 times more iodine and 50 times more iron than wheat. Dulse has purifying and tonic effects on the body. Another sea vegetable, Irish moss (chondrus crispus, carrageenan), contains electrolyte minerals, calcium, magnesium, sodium, and potassium. Its mucilaginous compounds help the body detoxify, boost metabolism, and strengthen hair, skin and nails.

The amount of the seaweed, kelp, or sea vegetable present in the dental powder product or the dental powder composition thereof may be from about 1.0 weight percent to about 30 weight percent, based on a dried, rehydrated, or fresh weight of the seaweed, kelp, or sea vegetable relative to a total weight of the dental powder product or the dental powder composition thereof. For example, the amount of the one or more seaweed, kelp, or sea vegetable present in the dental powder product or the dental powder composition thereof may be from about 1.0 weight percent, 25 weight percent, about 0.01 weight percent, about 20 weight percent, about 0.1 weight percent, about 15 weight percent, about 1 weight percent, about 5 weight percent, about 20 weight percent, or about 5 weight percent to about 15 weight percent, about 10 weight percent, or about 12 weight percent, based on a dried, rehydrated, or fresh weight of the seaweed, kelp, sea vegetable and/or parts thereof relative to a total weight of the dental powder product or the dental powder composition thereof.

In one embodiment, the amount of seaweed or kelp in the dental powder is between about 250 mg and 1000 mg, alternatively, between about 300 mg and about 900 mg, alternatively, between about 350 mg and about 800 mg, alternatively between about 400 mg and about 750 mg.

### Sodium Hexametaphosphate or Tartar Control Agents

In at least one implementation, the dental powder composition or the orally acceptable vehicle thereof includes a tartar control agent. In one embodiment, the tartar control agent is sodium hexametaphosphate, sodium acid phosphate (SAP), sodium acid pyrophosphate, or tripolyphosphate.

Sodium hexametaphosphate (SHMP) is a salt of composition Na6[(PO₃)₆ and is an anticalculus (tartar control) agent. Sodium hexametaphosphate of commerce is typically a mixture of metaphosphates (empirical formula:NaPO₃), of which the hexamer is one, and is usually the compound referred to by this name. Such a mixture is more correctly termed sodium polymetaphosphate. They are white solids that dissolve in water.

In other embodiments, sodium hexametaphosphate may be replaced by other tartar control agents, including but not limited to an anticalculus (tartar control) agent. Suitable anticalculus agents include, without limitation, phosphates and polyphosphates (for example pyrophosphates and tripolyphosphates), polyaminopropanesulfonic acid (AMPS), hexametaphosphate salts, zinc citrate trihydrate, polypeptides, polyolefin sulfonates, polyolefin phosphates, and diphosphonates.

The compositions of the disclosure thus may comprise phosphate salts in addition to the zinc phosphate. In particular embodiments, these salts are alkali phosphate salts, e.g., salts of alkali metal hydroxides or alkaline earth hydroxides, for example, sodium, potassium or calcium salts. “Phosphate” as used herein encompasses orally acceptable mono- and polyphosphates, for example, P1.6 phosphates, for example monomeric phosphates such as monobasic, dibasic or tribasic phosphate; and dimeric phosphates such as pyrophosphates; and multimeric phosphates, such as tripolyphosphates, tetraphosphates, hexaphosphates and hexametaphosphates (e.g., sodium hexametaphosphate). In particular examples, the selected phosphate is selected from alkali dibasic phosphate and alkali pyrophosphate salts, e.g, selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, and mixtures of any of two or more of these. In a particular embodiment, for example the compositions may comprise tetrasodium pyrophosphate in an amount of from 0.5 to 5% by weight, e.g., 1-3%, or 1-4%, or 2-4%), or 1-2% or about 2%, or about 4% by weight of the composition. In another embodiment, the compositions may comprise a mixture of tetrasodium pyrophosphate (TSPP) and sodium tripolyphosphate (STPP), e.g., in proportions of TSPP at from 0.5 to 5 wt. c′,/0, such as from 1 to 2 wt. % or 1 to 4 wt. % and STPP at from 0.5% to 6 wt. %, such as 1 to 4%, or 2 to 3% by weight of the composition. Such phosphates are provided in an amount effective to reduce erosion of the enamel, to aid in cleaning the teeth, and/or to reduce tartar buildup on the teeth, for example in an amount of from 0.2 to 10 wt. %, e.g., from 1 to 15 wt. %, by weight of the composition.

In one embodiment, the amount of Sodium Hexametaphosphate (HMP) in the dental powder is between about 50 mg and about 500 mg, alternatively, between about 75 mg and about 400 mg, alternatively, between about 100 mg and 300 mg, alternatively, between about 150 mg and 250 mg.

Botanical Blends and/or Parts Thereof

In one embodiment, the dental powder includes a botanical blend includes green tea and parsley. Green tea can have many benefits for oral health, including cavity prevention, gum health, and breath odor. Green tea's antioxidants kill bacteria and reduce the acidity of saliva and plaque, which can help prevent cavities. Green tea's flavonoids have anti-inflammatory properties that can help reduce gingivitis, the earliest stage of gum disease. Green tea's antibacterial properties can kill the microbes that cause bad breath. In one study, green tea outperformed mints, chewing gum, and parsley-seed oil at reducing smelly compounds in the mouth. In one embodiment, green tea is decaffeinated.

Parsley has many benefits for your teeth and oral health, including breath odor, antibacterial properties, antioxidants, and volatile oils. Parsley can help freshen your breath after a meal. Parsley's antibacterial properties can help prevent gum disease and tooth decay by eliminating harmful bacteria in your mouth. Parsley is high in vitamin K, which helps your body absorb calcium and remineralize teeth. Vitamin K is also important for bone health and wound healing. Parsley contains antioxidants like myricetin and apigenin, which have anti-inflammatory and cancer-fighting properties. Parsley contains volatile oils that are good for your oral health.

In other embodiments, the botanical blend may be a therapeutic agent, a cosmetic agent, chlorophyll and sodium copper chlorophyllin, and/or a decorative/aesthetic agent. In at least one implementation, the botanical blend may be a therapeutic agent capable of or configured to provide one or more therapeutic benefits. Illustrative therapeutic benefits may be or include, but are not limited to, anti-bleeding, anti-inflammatory, anti-bacteria, wound-healing, anti-oxidation, deodorization, sensorial related to oral comfort (e.g., oral hydration, gum soothing, etc.), or the like, or any combination thereof.

In another implementation, the botanical blend and/or parts thereof disposed in the orally acceptable vehicle may be a cosmetic agent and/or a decorative agent and may not or does not provide any therapeutic benefits. For example, the botanical blend may not provide any therapeutic benefits and may instead communicate or inform a consumer of one or more properties of the dental powder product or the dental powder composition thereof. The one or more properties of the dental powder product or the dental powder composition thereof that may be communicated to the consumer may be or include, but are not limited to, flavor, scent, brand, quality, emotional perception, mouthfeel, or the like, or any combination thereof. In another example, botanical blend and/or parts thereof may not provide any therapeutic benefits and may instead communicate or inform a consumer of the presence of one or more components in the dental powder product or the dental powder composition thereof. For example, the one or more botanical blend and/or parts thereof may communicate to the consumer of the presence of one or more elements or components from nature, one or more natural elements, and/or natural ingredients in the dental powder product or the dental powder composition thereof. In another example, the one or more botanical and/or parts thereof may be a decorative agent capable of or configured to provide an aesthetically pleasing and/or consumer acceptable decorative dental powder composition that enhances consumer experience and/or acceptance.

In yet another implementation, the botanical blend may provide at least one therapeutic benefit and may further inform the consumer of one or more properties of and/or the presence of one or more components in the dental powder composition. For example, the botanical blend may provide a therapeutic benefit and communicate to the consumer of one or more properties of the dental powder product or the dental powder composition thereof.

Theo botanical blend may be provided by any suitable botanical sources. Illustrative botanical sources may be or include, but are not limited to, any one or more of the botanical blend in the Kingdom Plantae, which may include any one or more botanical sources from any one or more of the following genus: Origanum, Thymus, Lavandula, Salvia, Melissa, Cuminum, Pelroselinum, Calendula, Tageles, Bowellia, Sambucus, Copaifera, Curcuma, Allium, Symphylum, Punica, Eulerpe, Sophora, Rheum, Fagopyrum, Camellia, Coplis, Hydraslis, Mahonia, Phellodendron, Berberis, Xanthorhiza, Lonicera, Vaccinium, Cinnamomum, VIZIS, Terminalia, Pinus, Albizia, Melia, Salvadora, Paullinia, Piper, Syzygium, Commiphora, Juglans, Sculellaria, and Magnolia. Illustrative botanical sources may also be or include, but are not limited to, roses, gardenias, jasmine, calendula, honeysuckle, cedar wood, aspen bark, willow bark, brahmi (Bacopa monnieri), green tea, grapes, pomegranate, echinacea, Centella asiatica, elderflower, Irish moss, mallow, soap bark, yucca, clary sage, oregano, thyme, or the like, or any combinations thereof.

The amount of the botanical blend present in the dental powder product or the dental powder composition thereof may be from about 0.001 weight percent to about 30 weight percent, based on a dried, rehydrated, or fresh weight of the botanical blend and/or parts thereof relative to a total weight of the dental powder product or the dental powder composition thereof. For example, the amount of the one or more botanical blend and/or parts thereof present in the dental powder product or the dental powder composition thereof may be from about 0.001 weight percent, 0.005 weight percent, about 0.01 weight percent, about 0.05 weight percent, about 0.1 weight percent, about 0.5 weight percent, about 1 weight percent, about 5 weight percent, about 10 weight percent, or about 15 weight percent to about 20 weight percent, about 25 weight percent, or about 30 weight percent, based on a dried, rehydrated, or fresh weight of the botanical blend and/or parts thereof relative to a total weight of the dental powder product or the dental powder composition thereof.

### Flavoring Agents

The dental powder composition or the orally acceptable vehicle thereof may also include one or more flavoring agents including mint flavor. Illustrative flavoring agents that may be utilized in the film forming composition may be or include, but are not limited to, essential oils and various flavoring aldehydes, esters, alcohols, and similar materials, as well as sweeteners such as sodium saccharin and/or sorbitol, peppermint flavor (e.g., OPTAMINT (R)), FREEZESTORM™, which is commercially available from Firmenich of Geneva, Switzerland, or the like, or mixtures or combinations thereof. Illustrative essential oils may include, but are not limited to, oils of spearmint, peppermint, wintergreen, sassafras, clove, sage, eucalyptus, marjoram, cinnamon, lemon, lime, grapefruit, catnip, and orange. Also useful are chemicals such as menthol, carvone, anethole, or the like, or mixtures or combinations thereof.

The amount or concentration of the one or more flavoring agents present in the dental powder product or the film forming composition thereof may vary widely. In at least one implementation, the amount of the one or more flavoring agents present may be from about 0.01 weight percent to about 5 weight percent, based on a total weight of the dental powder product or the film T forming composition thereof. For example, the amount of the one or more flavoring agents present may be from about 0.01 weight percent, about 1 weight percent, about 5 weight percent, about 10 weight percent, about 1.5 weight percent, about 2.0 weight percent, or about 2.5 weight percent to about 3.0 weight percent, about 3.5 weight percent, about 4.0 weight percent, about 4.5 weight percent, or about 5.0 weight percent.

Antimicrobial Agents and/or Preservatives

The dental powder composition or the orally acceptable vehicle thereof may also include mixed tocopherols, one or more antimicrobial agents and/or one or more preservatives. Illustrative antimicrobial agents may include, but are not limited to, methylisothiazolinone (MIT), sodium benzoate, potassium sorbate, halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC), phenolic antiseptics, hexetidine, octenidine, sanguinarine, povidone iodine, delmopinol, salifluor, other metal ions (e.g., stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol, and other piperidino derivatives, nicin preparations, chlorite salts, butylated hydroxyanisole (BHA) and butylated hydroxytoluene (BHT), and mixtures of any of the foregoing. In an exemplary implementation, the antibacterial agent includes cetylpyridinium chloride (CPC).

The amount of the mixed tocopherols or preservatives present in the dental powder product or the dental powder composition thereof may be from about 1.0 weight percent to about 0.30 weight percent, based on a dried, rehydrated, or fresh weight of the mixed tocopherols relative to a total weight of the dental powder product or the dental powder composition thereof. For example, the amount of the one or more probiotic, prebiotic, and/or postbiotic present in the dental powder product or the dental powder composition thereof may be from about 0.1 weight percent, 0.25 weight percent, about 0.01 weight percent, about 0.20 weight percent, about 0.1 weight percent, about 1.5 weight percent, about 1 weight percent, about 0.5 weight percent, about 0.20 weight percent, or about 0.5 weight percent to about 1.5 weight percent, about 1.0 weight percent, or about 1.2 weight percent, based on a dried, rehydrated, or fresh weight of the mixed tocopherols and/or parts thereof relative to a total weight of the dental powder product or the dental powder composition thereof.

### Orally Acceptable Vehicle

As discussed above, the compositions disclosed herein may be a dental powder composition. The dental powder composition may form at least a portion of or be used in one or more dental powder products. The dental powder composition may be, may include, or be combined with an orally acceptable vehicle to form the dental powder product (e.g., mouthwash). Illustrative dental powder products may include, but are not limited to, a toothpaste (dentifrice), a prophylactic paste, a tooth powder, a tooth polish, a tooth gel (e.g., a whitening gel), a chewing gum, a lozenge, a mouthwash, a whitening strip, a paint-on gel, varnish, veneer, and tube, syringe or dental tray comprising a gel or paste, or a gel or paste coated on an application support such as dental floss or a toothbrush (e.g., a manual, electric, sound, a combination thereof or ultrasound toothbrush), a tablet (e.g., effervescing tablets), granules, a caplet, wafers, films, beads, or the like. In a preferred implementation, the dental powder composition may be or may form at least a portion of a mouthwash.

In at least one implementation, the one or more kelp, sodium hexametaphosphate, a botanical blend, and a postbiotic and/or parts thereof may be dispersed and/or disposed in the orally acceptable vehicle. As used herein, the expression “orally acceptable vehicle” may refer to a suitable vehicle, ingredient, or combination of ingredients, which can be used to suspend, hold, mobilize, or otherwise contain the one or more kelp, sodium hexametaphosphate, a botanical blend, and a postbiotic and/or parts thereof in a safe and effective manner. For example, the orally acceptable vehicle may be a suitable solvent, and the one or more kelp, sodium hexametaphosphate, a botanical blend, and a postbiotic and/or parts thereof may be dispersed, dissolved, mixed, or otherwise contacted with the suitable solvent to prepare or form the dental powder product or the dental powder composition thereof. Illustrative solvents may be or include, but are not limited to, water, such as deionized or demineralized water, ethanol, methanol, isopropanol, butanol, water, acetone, methyl ethyl ketone, ethyl acetate, butyl acetate, cyclohexanone, cyclohexane, methylene chloride, chloroform, carbon tetrachloride, trichloroethylene, perchloroethylene, methyl glycol acetate, toluene, benzene, diethyl ether, benzyl alcohol, or the like, and mixtures or combinations thereof. In a preferred implementation, the orally acceptable vehicle includes water, more preferably demineralized water.

It should be appreciated that the orally acceptable vehicle may include materials such as, but not limited to, one or more antibacterial agents, fluoride ion sources, anticalculus agents, buffers, flavoring agents, sources of peroxide (e.g., hydrogen peroxide), alkali metal bicarbonate salts, thickening materials, antimicrobial agents, humectants, additional water, surfactants, titanium dioxide, cooling agents, coloring agents, or the like, or mixtures or combinations thereof. In at least one implementation, the one or more kelp, sodium hexametaphosphate, a botanical blend, and a postbiotic and/or parts thereof or the orally acceptable vehicle thereof may exclude any one or more of the aforementioned materials.

### Additional Ingredients

It should be appreciated by one having ordinary skill in the art, that the dental powder products and/or the dental powder compositions thereof may include other additional ingredients/components. For example, the dental powder products and/or the dental powder compositions thereof may include desensitizing agents, viscosity modifiers, diluents, surface active agents (e.g., emulsifiers, foam modulators, etc.), pH modifying agents (e.g., acids and bases), mouth feel agents, sweetening agents, colorants/dies, preservatives, or the like, or combinations or mixtures thereof. It should further be appreciated by one having ordinary skill in the art that while general attributes of each of the above categories of materials may differ, there may be some common attributes and any given material may serve multiple purposes within two or more of such categories of materials.

All ingredients for use in the compositions described herein should be orally acceptable. As used herein, “orally acceptable” may refer to any ingredient that is present in a composition as described in an amount and form that does not render the composition unsafe for use in the oral cavity.

### Method

The present disclosure may provide a method for preparing a dental powder product or a dental powder composition thereof. The method may include preparing an orally acceptable vehicle for the dental powder composition. The method may also include contacting one or more kelp, sodium hexametaphosphate, a botanical blend, and a postbiotic and/or parts thereof with the orally acceptable vehicle. In at least one implementation, the one or more kelp, sodium hexametaphosphate, a botanical blend, and a postbiotic and/or parts thereof may be heated or placed in a heated medium, such as water, to solubilize or facilitate the solubilization of a portion of any anthocyanin contained in the kelp, sodium hexametaphosphate, a botanical blend, and a postbiotic and/or the parts thereof. In at least one implementation, the method may include suspending or substantially distributing the one or more kelp, sodium hexametaphosphate, a botanical blend, and a postbiotic and/or parts thereof in the orally acceptable vehicle. Suspending or substantially distributing the one or more kelp, sodium hexametaphosphate, a botanical blend, and a postbiotic and/or parts thereof in the orally acceptable vehicle may include adding one or more thickeners or thickening agents to the orally acceptable vehicle. In another implementation, the dental powder composition may be free or substantially free of any thickeners or thickening agents; and thus, the method may exclude adding any thickeners or thickening agents.

In one embodiment, the dental powder product may include an inactive ingredient for anti-caking/processing agent, such as bentonite clay. In another embodiment, the dental powder product will not include the anti-caking/processing agent and include a desiccant pack in the packaging.

The present disclosure may provide a method for treating, reducing, or ameliorating one or more oral conditions in a user, human, or animal subject in need thereof with the dental powder compositions including the one or more kelp, sodium hexametaphosphate, a botanical blend, and a postbiotic and/or parts thereof. As used herein “animal subject” may include higher order non-human mammals such as canines, felines, and horses. The one or more oral conditions may include any oral conditions treatable with any one or more therapeutic benefits of the kelp, sodium hexametaphosphate, a botanical blend, and a postbiotic and/or parts thereof. As discussed above, the one or more therapeutic benefits may be or include, but are not limited to, beath odor, tooth whiteness, plaque and tartar, gum health, and overall dental health related to oral comfort (e.g., oral hydration, gum soothing, etc.), or the like, or any combination thereof. The method may include contacting the dental powder composition with surfaces of the oral cavity. For example, the method may include rinsing, swishing, or gargling the dental powder composition in the oral cavity to contact the dental powder composition with surfaces of the oral cavity. The method may include contacting the dental powder composition with surfaces of the oral cavity at least once per day, at least twice per day, at least three times per week, or more.

The method of applying the dental powder to pet food comprises administering the dental powder twice a day to a food product for an extended time period. In one embodiment, the time period is about 8 weeks.

TABLE 1
Dental Powder Composition
Product Formula

| | | | | | | |
|---|---|---|---|---|---|---|
| | | Label Claim | RM | | Input | |
| PH RM Code | Ingredient | (mg) | Assay % | Overage % | (mg) | % |

Actives

| | | | | | |
|---|---|---|---|---|---|
| — | Kelp | 565 | — | 565.00 | 11.300% |
| | [Brown Algae | | | | |
| | (Ascophyllum nodosum)] | | | | |
| — | Sodium | 150 | 95% | 157.89 | 3.158% |
| | Hexametaphosphate | | | | |
| — | Parsley | 100 | — | 60.00 | 1.200% |
| — | Decaffeinated | | — | 40.00 | 0.800% |
| | Green Tea Leaf Powder | | | | |
| — | Postbiotic (L. plantarum) | 10 | 100B HT | 10.00 | 0.200% |
| | | | cells/g | | |

Inactives

| | | | | | | |
|---|---|---|---|---|---|---|
| — | Brown Rice Flour | — | — | | 3337.11 | 66.742% |
| — | Hydrolyzed Yeast | — | — | | 250.00 | 5.000% |
| — | Pork Liver | — | — | | 400.00 | 8.000% |
| — | Bentonite | — | — | | 100.00 | 2.000% |
| — | Natural Mint Flavor | — | — | | 75.00 | 1.500% |
| — | Mixed Tocopherols | — | — | | 5.00 | 0.100% |
| | | | | Total | 5000.00 | 100.000% |

Label Information
Scoop Size 2.5 grams Serving Size 2 Scoops (5 grams)/25 lbs

| | |
|---|---|
| Active Ingredients | |
| Kelp | 565 mg |
| Sodium Hexametaphosphate | 150 mg |
| Fresh Breath Blend | 100 mg |
| (Decaffeinated Green Tea & Parsley) | |
| Postbiotic (L. plantarum) | 10 mg |

Inactive Ingredients

Bentonite, Brown Rice, Hydrolyzed Yeast, Mixed Tocopherols
(Preservative), Natural Mint Flavor, Natural Pork Flavor.

Directions for Use

For pets <25 lbs, mix 1 scoop. For pets 25-75 lbs, mix 2 scoops in a bowl at meal time.
For pets >75 lbs, mix 3 scoops. If desired, add a splash of water to adjust texture.

### Formula Embodiment

In one embodiment, the serving amount of the dental powder is 2.5 g for animals less than 25 lbs, 2.5 g for animals between 25-75 lbs, and 2.5 g for animals for animals weighing more than 75 lbs. The serving amount of the dental powder is 2.5 g for animals less than 25 lbs, 5.0 g for animals between 25-75 lbs, and 7.5-8.0 g for animals for animals weighing more than 75 lbs.

In one embodiment, the dental powder formula was administered to a pet for about 8 weeks, as shown in FIG. 1B. FIG. 1B is a graph of the % of pet parents who saw improvement in the dog's breath odor, tooth whiteness, visible plaque and tartar, gum health and overall dental health from week 1 to week 8. As demonstrated, the dental powder composition improved breath odor from baseline 3.4 day 0 to 6.7 after 8 weeks of use of the dental powder composition. Tooth whiteness also improved from baseline 3.4 at day 0 to 6.5 after 8 weeks of use of the dental powder composition. Overall dental health improved from baseline of 3.6 at day 0 to 6.6 after 8 weeks of use the dental powder. Gum health also improved 75% after 8 weeks of use of the dental powder composition.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Canine Preference Results: Pet Honesty Fresh Breath Dental Powder Composition (PH Dental) vs PetLabCo ProBright Dental Powder (PetLab Dental)

### Consumption Summary

The total amount of food consumed by 60 dogs during a 5 day time period of this test was 61,392 Grams and as follows: Diet A as Chow+PH Dental: 50,949 Grams or 83.0%. Diet B as Chow+PetLab Dental: 10,443 Grams or 17.0%. PH Dental is the dental powder composition of the present invention. PH Dental composition includes Brown Algae (Ascophyllum nodosum), Sodium Hexametaphosphate, Fresh Breath Blend (Decaffeinated Green Tea & Parsley), Postbiotic (L. plantarum) as well as Brown Rice Flour, Pork Liver, Bentonite, Natural Mint Flavor, Mixed Tocopherols. The PetLab Dental powder includes Lactobacillus helveticus, L. rhamnosus, and Bifidobacterium longum, brown algae, sodium hexametaphosphate, rosemary leaf extract, green tea, and perilla seed extract, as well as Dried Brewer's Yeast, Spinach Powder, Yeast. There is a 4.88 to 1 consumption ratio of Chow+PH Dental to Chow+PetLab Dental, as indicated below.

Using a Wilcoxon Signed Rank test there is significant reason to suspect a consumption difference between the two Diets (P=0.0000). A 2-way analysis of variance (2-Way ANOVA) indicates that there is significant reason to suspect a difference in consumption of the 2 diets when the variance in consumption of the dogs is accounted for (F=143.356, P=0.0000).

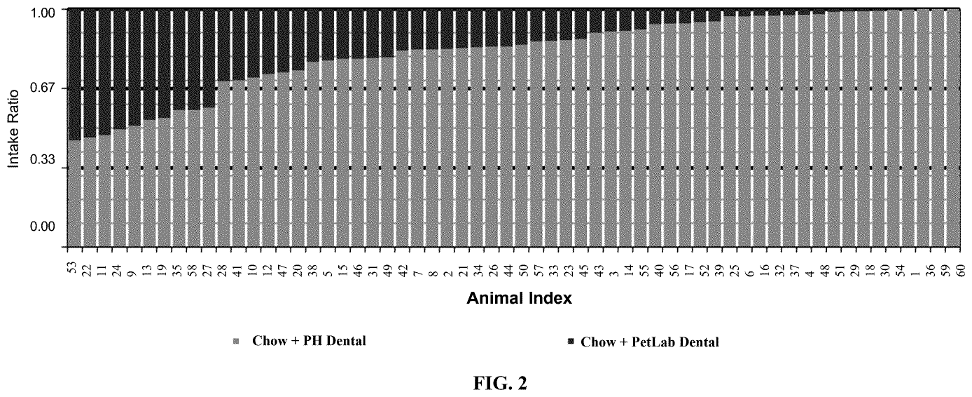

The Intake Ratio Chart is shown in FIG. 2, where the individual Intake Ratios for each of the 60 dogs are sorted in ascending order for Chow+PH Dental. The average of the 60 individual Intake Ratios for each Diet are as follows: Diet A as Chow+PH Dental:Average IR=0.822. Diet B as Chow+PetLab Dental:Average IR=0.178.

### Preference By First Choice

Over all 5 days Chow+PH Dental was chosen first on 222 occasions and Chow+PetLab Dental was chosen first on 78 occasions. A significant First Choice is detected and tends to favor Chow+PH Dental. The Chi² probability is p=0.0000.

Over all 5 days 17 dogs chose Chow+PH Dental first every day and no dogs chose Chow+PetLab Dental first every day, the other 43 were somewhat undecided. There is significant reason to show that this result is not random as a definite preference is detected. The Chi² probability is p=0.0000.

### Daily Consumption Summary

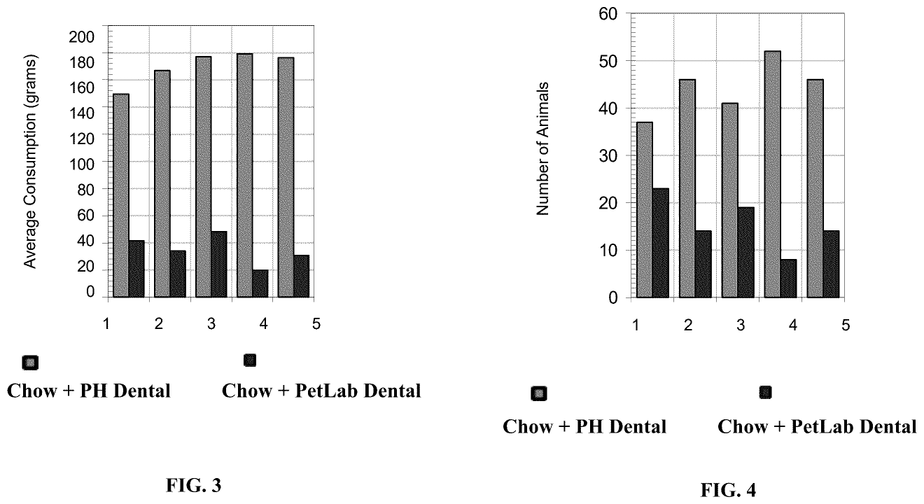

The average daily consumption of each diet is shown in FIG. 3 and Table 2 for each of the 5 days. The consumption of each diet is compared using a separate paired t-Test within each day (60 dogs by 2 diets) to assess significance in consumption difference for that day.

TABLE 2
Average Daily Consumption Data

| Day | Diet A | Diet B | Total | P of t | Significant |
|---|---|---|---|---|---|
| 1 | 149.6 | 41.5 | 191.1 | 0.0000 | YES! |
| 2 | 166.9 | 33.9 | 200.8 | 0.0000 | YES! |
| 3 | 177.1 | 48.2 | 225.4 | 0.0000 | YES! |
| 4 | 179.2 | 19.7 | 198.9 | 0.0000 | YES! |
| 5 | 176.4 | 30.7 | 207.1 | 0.0000 | YES! |

### Daily Preference By First Choice

The diet of First Choice preference is scored daily for each dog and the counts are shown in FIG. 4 and Table 3. A Chi² test compares the daily counts for Chow+ PH Dental and Chow+PetLab Dental to the counts that might have been expected if there was no First-Choice difference in the two diets. In this case that would be a 30:30 ratio or both diets chosen equally. Now, a Significant result (YES!) here indicates that these products were not chosen equally. Whereas a non-Significant result (NO) means that there is no evidence to suspect a First-Choice difference.

TABLE 3
Daily First Choice Counts

| Day | Diet A | Diet B | P of Chi² | Significant |
|---|---|---|---|---|
| 1 | 37 | 23 | 0.0707 | NO |
| 2 | 46 | 14 | 0.0000 | YES! |
| 3 | 41 | 19 | 0.0045 | YES! |
| 4 | 52 | 8 | 0.0000 | YES! |
| 5 | 46 | 14 | 0.0000 | YES! |

### Preference Scoring by Intake Ratio

It is a known contention that an animal shows a clear preference by consuming twice as much of one diet as the other. This is measured as a 2:1 consumption ratio or a 0.6667 Intake Ratio.

Scoring is achieved by reviewing the average intake ratios for each dog in the test and scoring one point for the Diet with an Intake Ratio greater than or equal to 0.6667.

Using the 2:1 consumption criteria in the Intake Preference chart to the right, 50 dogs prefer Chow+PH Dental, no dogs prefer Chow+PetLab Dental and the other 10 dogs show no Preference, as shown in FIG. 5.

### Methods and Procedures

The kennel facility is registered with the USDA No. 23-R-0126 under the Animal Welfare Act. The kennel had a 12-hour-light/12-hour-dark cycle. Every attempt was made to keep temperature ranges within targeted conditions (from 50° to 80° F.) in accordance with the Animal Welfare Act. Cages and feed bowls were cleaned daily and sanitized in accordance with the Animal

### Welfare Act.

The purpose of the study was to assess the palatability of Chow+PH Dental and Chow+PetLab Dental. Sixty male and female Beagles identified by ear tattoo and cage number were presented the test diets on an individual basis. Two stainless steel bowls, each containing approximately 405 grams of diet, were offered once daily for 5 days. To create each diet, 5 grams of the designated powder were mixed into 400 grams of Purina Dog Chow. Bowl placement was reversed daily and both bowls were presented for 30 minutes. If one diet was completely consumed prior to the end of the 30 minutes, both bowls were removed. Food consumption and first choice preference were recorded for each dog.

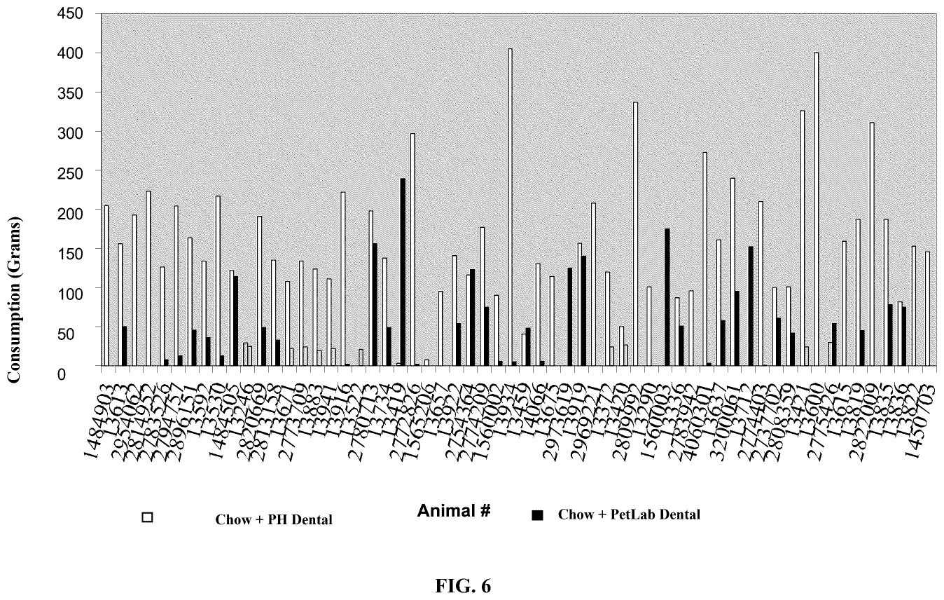

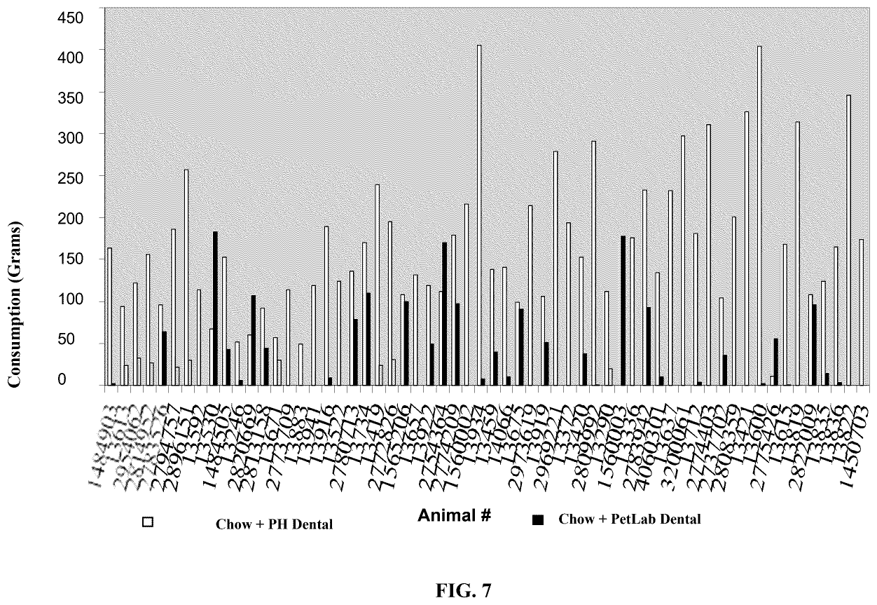

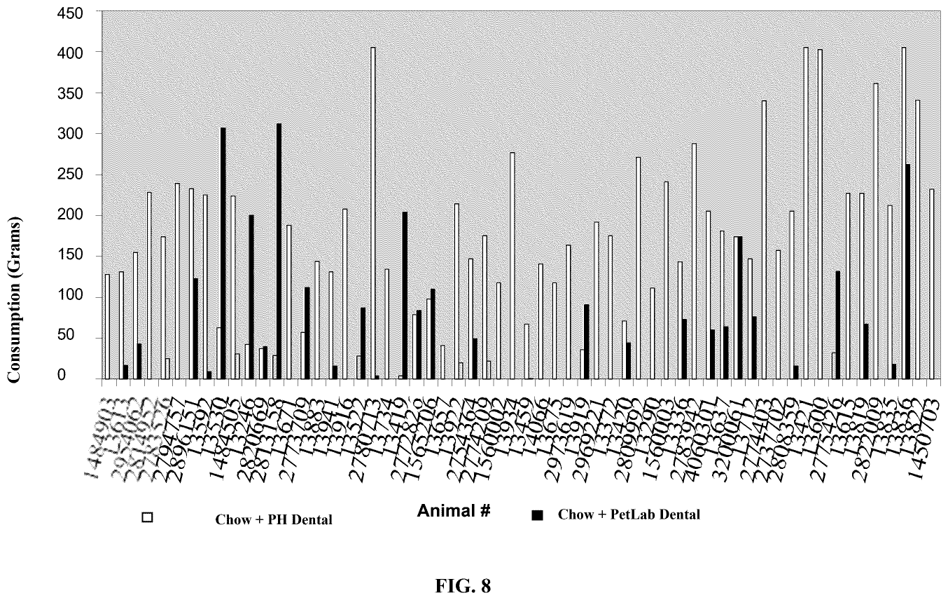

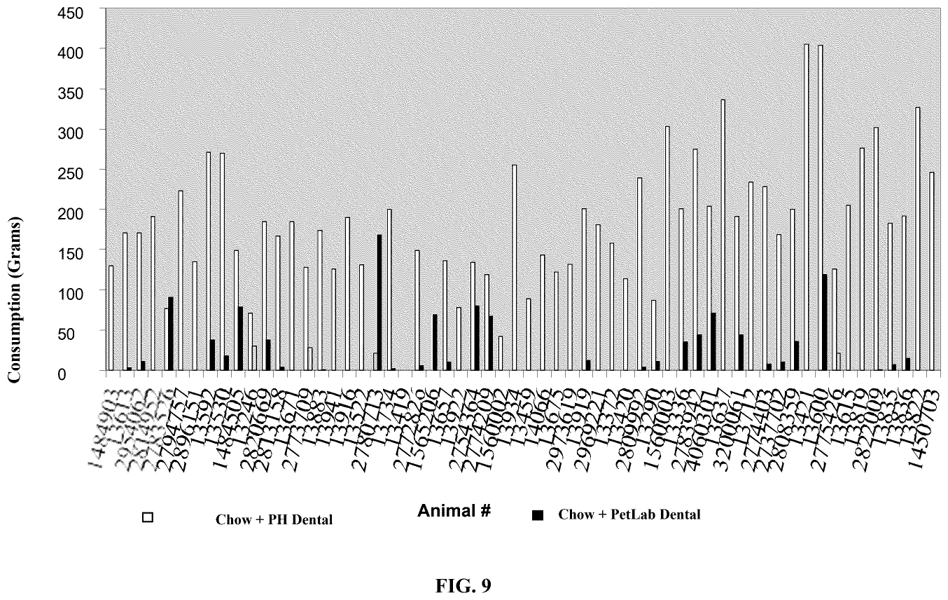

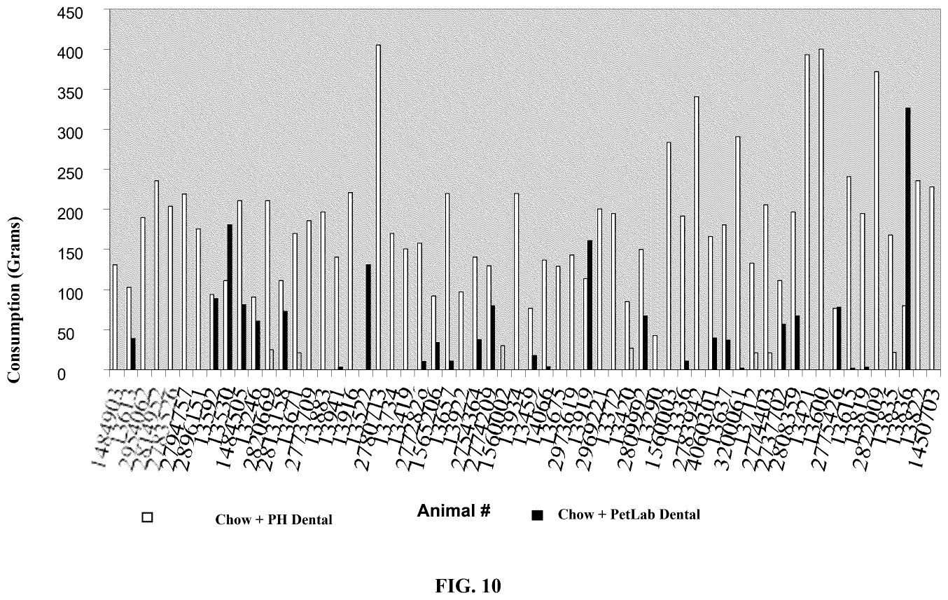

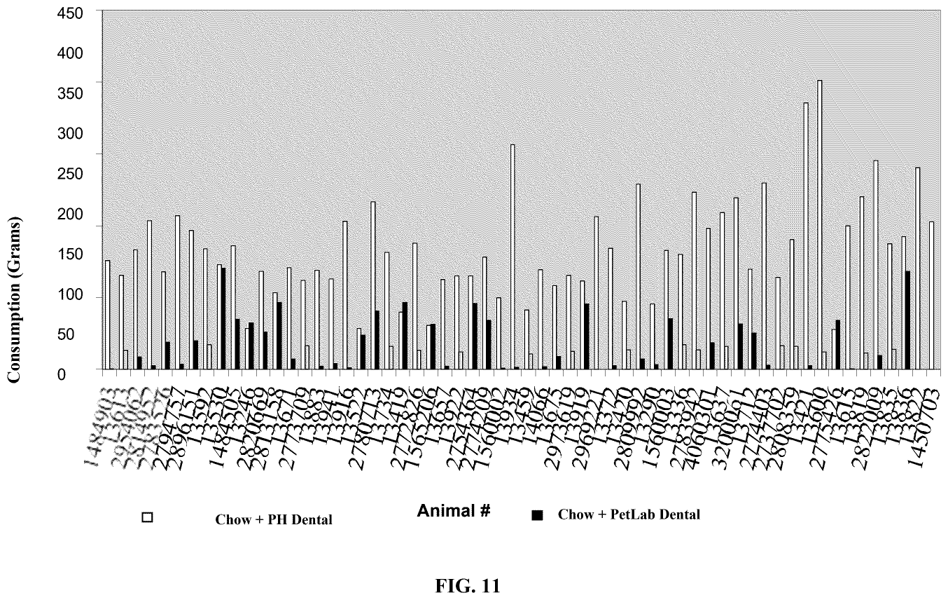

FIG. 6 is a graph of the day 1 food consumption and first choice data for each dog. FIG. 7 is a graph of the day 2 food consumption and first choice data for each dog. FIG. 8 is a graph of the day 3 food consumption and first choice data for each dog. FIG. 9 is a graph of the day 4 food consumption and first choice data for each dog. FIG. 10 is a graph of the day 5 food consumption and first choice data for each dog. FIG. 11 is a graph of the total food consumption and first choice data for each dog.

TABLE 4
Day 1 food consumption and First Choice Data

| | | Grams of Diet Chow + | | | | Grams of Diet Chow + | | | | |
|---|---|---|---|---|---|---|---|---|---|---|
| | | PH Dental | | | | Pet Lab Dental | | | | |
| | | A | A | A | | B | B | B | | |
| | | Weigh | Weigh | Total | First | Weigh | Weigh | Total | First | Intake |
| | Dog | In | Out | Consumed | Choice | In | Out | Consumed | Choice | Ratio |
| 1 | 1484903 | 677 | 472 | 205 | * | 548 | 548 | 0 | | 1.00 |
| 2 | 13613 | 550 | 394 | 156 | | 633 | 583 | 50 | * | 0.76 |
| 3 | 2954062 | 553 | 360 | 193 | * | 592 | 592 | 0 | | 1.00 |
| 4 | 2814952 | 630 | 407 | 223 | * | 582 | 582 | 0 | | 1.00 |
| 5 | 2783526 | 558 | 432 | 126 | | 600 | 592 | 8 | * | 0.94 |
| 6 | 2794757 | 615 | 411 | 204 | * | 622 | 609 | 13 | | 0.94 |
| 7 | 2896151 | 545 | 381 | 164 | * | 626 | 580 | 46 | | 0.78 |
| 8 | 13592 | 607 | 473 | 134 | * | 625 | 589 | 36 | | 0.79 |
| 9 | 13530 | 602 | 385 | 217 | * | 619 | 606 | 13 | | 0.94 |
| 10 | 1484505 | 610 | 488 | 122 | * | 619 | 505 | 114 | | 0.52 |
| 11 | 13246 | 611 | 582 | 29 | * | 620 | 595 | 25 | | 0.54 |
| 12 | 2820669 | 599 | 408 | 191 | * | 585 | 536 | 49 | | 0.80 |
| 13 | 2813158 | 604 | 469 | 135 | * | 597 | 564 | 33 | | 0.80 |
| 14 | 13671 | 610 | 502 | 108 | * | 579 | 557 | 22 | | 0.83 |
| 15 | 2773709 | 605 | 471 | 134 | * | 577 | 553 | 24 | | 0.85 |
| 16 | 13883 | 628 | 504 | 124 | * | 582 | 562 | 20 | | 0.86 |
| 17 | 13941 | 623 | 512 | 111 | * | 636 | 614 | 22 | | 0.83 |
| 18 | 13916 | 625 | 403 | 222 | * | 576 | 574 | 2 | | 0.99 |
| 19 | 13522 | 623 | 623 | 0 | | 623 | 602 | 21 | * | 0.00 |
| 20 | 2780713 | 622 | 424 | 198 | | 623 | 467 | 156 | * | 0.56 |
| 21 | 13734 | 603 | 465 | 138 | | 622 | 573 | 49 | * | 0.74 |
| 22 | 13419 | 636 | 633 | 3 | * | 613 | 374 | 239 | | 0.01 |
| 23 | 2772826 | 578 | 281 | 297 | * | 621 | 619 | 2 | | 0.99 |
| 24 | 1565206 | 621 | 613 | 8 | | 619 | 619 | 0 | * | 1.00 |
| 25 | 13657 | 576 | 48 | 95 | * | 617 | 617 | 0 | | 1.00 |
| 26 | 13922 | 622 | 48 | 141 | | 629 | 575 | 54 | * | 0.72 |
| 27 | 2754364 | 623 | 507 | 116 | | 614 | 491 | 123 | * | 0.49 |
| 28 | 2774209 | 619 | 442 | 177 | | 617 | 542 | 75 | * | 0.70 |
| 29 | 1560002 | 633 | 543 | 90 | * | 623 | 617 | 6 | | 0.94 |
| 30 | 13934 | 637 | 232 | 405 | * | 618 | 613 | 5 | | 0.99 |
| 31 | 13459 | 577 | 536 | 41 | | 613 | 565 | 48 | * | 0.46 |
| 32 | 14066 | 576 | 445 | 131 | * | 621 | 615 | 6 | | 0.96 |
| 33 | 13675 | 630 | 516 | 114 | | 623 | 623 | 0 | * | 1.00 |
| 34 | 2973619 | 634 | 634 | 0 | | 623 | 498 | 125 | * | 0.00 |
| 35 | 13919 | 638 | 481 | 157 | * | 619 | 479 | 140 | | 0.53 |
| 36 | 2969221 | 629 | 421 | 208 | * | 615 | 615 | 0 | | 1.00 |
| 37 | 13372 | 578 | 458 | 120 | * | 624 | 600 | 24 | | 0.83 |
| 38 | 13420 | 581 | 53 | 50 | * | 616 | 589 | 27 | | 0.65 |
| 39 | 2809992 | 630 | 293 | 337 | * | 615 | 615 | 0 | | 1.00 |
| 40 | 13290 | 628 | 527 | 101 | | 626 | 626 | 0 | * | 1.00 |
| 41 | 1560003 | 629 | 629 | 0 | | 604 | 429 | 175 | * | 0.00 |
| 42 | 13336 | 593 | 506 | 87 | | 605 | 554 | 51 | * | 0.63 |
| 43 | 2783942 | 582 | 486 | 96 | * | 608 | 608 | 0 | | 1.00 |
| 44 | 4060301 | 582 | 309 | 273 | * | 623 | 620 | 3 | | 0.99 |
| 45 | 13637 | 580 | 419 | 161 | | 609 | 551 | 58 | * | 0.74 |
| 46 | 3200061 | 577 | 337 | 240 | * | 606 | 511 | 95 | | 0.72 |
| 47 | 13712 | 580 | 580 | 0 | | 609 | 457 | 152 | * | 0.00 |
| 48 | 2774403 | 578 | 368 | 210 | | 601 | 600 | 1 | * | 1.00 |
| 49 | 2737702 | 575 | 475 | 100 | * | 608 | 547 | 61 | | 0.62 |
| 50 | 2808359 | 580 | 479 | 101 | | 608 | 566 | 42 | * | 0.71 |
| 51 | 13421 | 586 | 260 | 326 | * | 580 | 556 | 24 | | 0.93 |
| 52 | 13600 | 576 | 176 | 400 | * | 581 | 581 | 0 | | 1.00 |
| 53 | 2775426 | 579 | 549 | 30 | | 576 | 522 | 54 | * | 0.36 |
| 54 | 13615 | 591 | 432 | 159 | * | 631 | 631 | 0 | | 1.00 |
| 55 | 13819 | 579 | 392 | 187 | | 617 | 572 | 45 | * | 0.81 |
| 56 | 2822009 | 617 | 306 | 311 | | 642 | 642 | 0 | * | 1.00 |
| 57 | 13835 | 632 | 445 | 187 | * | 621 | 543 | 78 | | 0.71 |
| 58 | 13836 | 576 | 494 | 82 | | 631 | 556 | 75 | * | 0.52 |
| 59 | 13822 | 640 | 487 | 153 | * | 639 | 639 | 0 | | 1.00 |
| 60 | 1450703 | 583 | 437 | 146 | * | 641 | 641 | 0 | | 1.00 |
| | Total: | 36161 | 27187 | 8974 | 37 | 36692 | 34201 | 2491 | 23 | 45.45 |
| | Average: | 602.7 | 453.1 | 149.6 | | 611.5 | 570.0 | 41.5 | | 0.76 |

The underlined numbers indicate all of the diet offered was consumed. 3.60 to 1 consumption ratio for Chow+PH Dental to Chow+PetLab Dental. 1.61 to 1 First Choice ratio for Chow+PH Dental to Chow+PetLab Dental.

TABLE 5
Day 2 Food Consumption and First Choice Data

| | | Grams of Diet Chow + | | | | Grams of Diet Chow + | | | | |
|---|---|---|---|---|---|---|---|---|---|---|
| | | PH Dental | | | | Pet Lab Dental | | | | |
| | | A | A | A | | B | B | B | | |
| | | Weigh | Weigh | Total | First | Weigh | Weigh | Total | First | Intake |
| | Dog | In | Out | Consumed | Choice | In | Out | Consumed | Choice | Ratio |
| 1 | 1484903 | 677 | 513 | 164 | | 549 | 547 | 2 | * | 0.99 |
| 2 | 13613 | 550 | 456 | 94 | * | 634 | 610 | 24 | | 0.80 |
| 3 | 2954062 | 553 | 431 | 122 | * | 592 | 559 | 33 | | 0.79 |
| 4 | 2814952 | 630 | 474 | 156 | * | 583 | 556 | 27 | | 0.85 |
| 5 | 2783526 | 559 | 463 | 96 | * | 600 | 536 | 64 | | 0.60 |
| 6 | 2794757 | 615 | 429 | 186 | * | 623 | 601 | 22 | | 0.89 |
| 7 | 2896151 | 546 | 289 | 257 | * | 626 | 596 | 30 | | 0.90 |
| 8 | 13592 | 607 | 493 | 114 | * | 625 | 625 | 0 | | 1.00 |
| 9 | 13530 | 602 | 535 | 67 | | 619 | 436 | 183 | * | 0.27 |
| 10 | 1484505 | 611 | 458 | 153 | * | 619 | 576 | 43 | | 0.78 |
| 11 | 13246 | 610 | 558 | 52 | * | 621 | 615 | 6 | | 0.90 |
| 12 | 2820669 | 599 | 539 | 60 | | 585 | 478 | 107 | * | 0.36 |
| 13 | 2813158 | 604 | 512 | 92 | | 597 | 553 | 44 | * | 0.68 |
| 14 | 13671 | 611 | 554 | 57 | | 579 | 549 | 30 | * | 0.66 |
| 15 | 2773709 | 604 | 490 | 114 | * | 578 | 578 | 0 | | 1.00 |
| 16 | 13883 | 628 | 579 | 49 | * | 583 | 583 | 0 | | 1.00 |
| 17 | 13941 | 624 | 505 | 119 | * | 636 | 636 | 0 | | 1.00 |
| 18 | 13916 | 625 | 436 | 189 | | 577 | 568 | 9 | * | 0.95 |
| 19 | 13522 | 623 | 499 | 124 | * | 624 | 624 | 0 | | 1.00 |
| 20 | 2780713 | 622 | 486 | 136 | * | 623 | 544 | 79 | | 0.63 |
| 21 | 13734 | 603 | 433 | 170 | * | 622 | 512 | 110 | | 0.61 |
| 22 | 13419 | 636 | 397 | 239 | * | 613 | 589 | 24 | | 0.91 |
| 23 | 2772826 | 579 | 384 | 195 | * | 622 | 591 | 31 | | 0.86 |
| 24 | 1565206 | 621 | 513 | 108 | * | 619 | 519 | 100 | | 0.52 |
| 25 | 13657 | 577 | 445 | 132 | * | 618 | 618 | 0 | | 1.00 |
| 26 | 13922 | 622 | 503 | 119 | | 631 | 582 | 49 | * | 0.71 |
| 27 | 2754364 | 623 | 511 | 112 | | 614 | 444 | 170 | * | 0.40 |
| 28 | 2774209 | 621 | 442 | 179 | | 617 | 520 | 97 | * | 0.65 |
| 29 | 1560002 | 634 | 418 | 216 | * | 623 | 623 | 0 | | 1.00 |
| 30 | 13934 | 638 | 233 | 405 | * | 618 | 610 | 8 | | 0.98 |
| 31 | 13459 | 578 | 440 | 138 | | 614 | 574 | 40 | * | 0.78 |
| 32 | 14066 | 577 | 436 | 141 | * | 619 | 609 | 10 | | 0.93 |
| 33 | 13675 | 631 | 532 | 99 | * | 621 | 530 | 91 | | 0.52 |
| 34 | 2973619 | 634 | 420 | 214 | * | 621 | 621 | 0 | | 1.00 |
| 35 | 13919 | 638 | 532 | 106 | * | 621 | 570 | 51 | | 0.68 |
| 36 | 2969221 | 630 | 351 | 279 | * | 616 | 616 | 0 | | 1.00 |
| 37 | 13372 | 579 | 385 | 194 | * | 623 | 623 | 0 | | 1.00 |
| 38 | 13420 | 581 | 428 | 153 | * | 616 | 578 | 38 | | 0.80 |
| 39 | 2809992 | 630 | 339 | 291 | * | 616 | 615 | 1 | | 1.00 |
| 40 | 13290 | 628 | 516 | 112 | * | 626 | 606 | 20 | | 0.85 |
| 41 | 1560003 | 628 | 628 | 0 | | 603 | 425 | 178 | * | 0.00 |
| 42 | 13336 | 593 | 417 | 176 | * | 605 | 605 | 0 | | 1.00 |
| 43 | 2783942 | 582 | 349 | 233 | * | 609 | 516 | 93 | | 0.71 |
| 44 | 4060301 | 581 | 447 | 134 | | 623 | 613 | 10 | * | 0.93 |
| 45 | 13637 | 580 | 348 | 232 | * | 609 | 609 | 0 | | 1.00 |
| 46 | 3200061 | 578 | 281 | 297 | * | 606 | 606 | 0 | | 1.00 |
| 47 | 13712 | 580 | 399 | 181 | * | 609 | 605 | 4 | | 0.98 |
| 48 | 2774403 | 577 | 266 | 311 | * | 601 | 601 | 0 | | 1.00 |
| 49 | 2737702 | 575 | 471 | 104 | * | 610 | 574 | 36 | | 0.74 |
| 50 | 2808359 | 580 | 379 | 201 | * | 609 | 609 | 0 | | 1.00 |
| 51 | 13421 | 587 | 261 | 326 | * | 579 | 579 | 0 | | 1.00 |
| 52 | 13600 | 577 | 173 | 404 | * | 580 | 578 | 2 | | 1.00 |
| 53 | 2775426 | 580 | 569 | 11 | * | 576 | 520 | 56 | | 0.16 |
| 54 | 13615 | 591 | 423 | 168 | * | 631 | 630 | 1 | | 0.99 |
| 55 | 13819 | 580 | 266 | 314 | * | 617 | 617 | 0 | | 1.00 |
| 56 | 2822009 | 616 | 508 | 108 | | 641 | 545 | 96 | * | 0.53 |
| 57 | 13835 | 632 | 508 | 124 | | 621 | 607 | 14 | * | 0.90 |
| 58 | 13836 | 576 | 411 | 165 | * | 633 | 630 | 3 | | 0.98 |
| 59 | 13822 | 642 | 296 | 346 | * | 639 | 639 | 0 | | 1.00 |
| 60 | 1450703 | 582 | 408 | 174 | * | 640 | 640 | 0 | | 1.00 |
| | Total: | 36177 | 26165 | 10012 | 46 | 36704 | 34668 | 2036 | 14 | 49.15 |
| | Average: | 603.0 | 436.1 | 166.9 | | 611.7 | 577.8 | 33.9 | | 0.82 |

The underlined numbers indicate all of the diet offered was consumed. 4.92 to 1 consumption ratio for Chow+PH Dental to Chow+PetLab Dental. 3.29 to 1 First Choice ratio for Chow+PH Dental to Chow+PetLab Dental.

TABLE 6
Day 3 Food Consumption and First Choice Data

| | | Grams of Diet Chow + | | | | Grams of Diet Chow + | | | | |
|---|---|---|---|---|---|---|---|---|---|---|
| | | PH Dental | | | | Pet Lab Dental | | | | |
| | | A | A | A | | B | B | B | | |
| | | Weigh | Weigh | Total | First | Weigh | Weigh | Total | First | Intake |
| | Dog | In | Out | Consumed | Choice | In | Out | Consumed | Choice | Ratio |
| 1 | 1484903 | 677 | 549 | 128 | * | 550 | 550 | 0 | | 1.00 |
| 2 | 13613 | 551 | 420 | 131 | * | 634 | 617 | 17 | | 0.89 |
| 3 | 2954062 | 552 | 397 | 155 | | 592 | 549 | 43 | * | 0.78 |
| 4 | 2814952 | 630 | 402 | 228 | * | 583 | 583 | 0 | | 1.00 |
| 5 | 2783526 | 559 | 385 | 174 | | 600 | 575 | 25 | * | 0.87 |
| 6 | 2794757 | 615 | 376 | 239 | * | 623 | 623 | 0 | | 1.00 |
| 7 | 2896151 | 547 | 314 | 233 | | 627 | 504 | 123 | * | 0.65 |
| 8 | 13592 | 607 | 382 | 225 | * | 626 | 617 | 9 | | 0.96 |
| 9 | 13530 | 602 | 539 | 63 | | 620 | 313 | 307 | * | 0.17 |
| 10 | 1484505 | 610 | 386 | 224 | * | 619 | 588 | 31 | | 0.88 |
| 11 | 13246 | 611 | 569 | 42 | | 622 | 422 | 200 | * | 0.17 |
| 12 | 2820669 | 600 | 563 | 37 | | 585 | 545 | 40 | * | 0.48 |
| 13 | 2813158 | 604 | 575 | 29 | * | 598 | 286 | 312 | | 0.09 |
| 14 | 13671 | 611 | 423 | 188 | * | 579 | 579 | 0 | | 1.00 |
| 15 | 2773709 | 606 | 549 | 57 | * | 577 | 465 | 112 | | 0.34 |
| 16 | 13883 | 629 | 485 | 144 | * | 582 | 582 | 0 | | 1.00 |
| 17 | 13941 | 623 | 492 | 131 | | 636 | 620 | 16 | * | 0.89 |
| 18 | 13916 | 626 | 418 | 208 | * | 577 | 577 | 0 | | 1.00 |
| 19 | 13522 | 624 | 596 | 28 | | 623 | 536 | 87 | * | 0.24 |
| 20 | 2780713 | 622 | 217 | 405 | * | 623 | 619 | 4 | | 0.99 |
| 21 | 13734 | 602 | 468 | 134 | * | 622 | 622 | 0 | | 1.00 |
| 22 | 13419 | 636 | 632 | 4 | | 612 | 408 | 204 | * | 0.02 |
| 23 | 2772826 | 579 | 500 | 79 | | 619 | 535 | 84 | * | 0.48 |
| 24 | 1565206 | 621 | 523 | 98 | | 618 | 508 | 110 | * | 0.47 |
| 25 | 13657 | 577 | 536 | 41 | * | 616 | 616 | 0 | | 1.00 |
| 26 | 13922 | 621 | 407 | 214 | | 630 | 610 | 20 | * | 0.91 |
| 27 | 2754364 | 622 | 475 | 147 | | 614 | 565 | 49 | * | 0.75 |
| 28 | 2774209 | 619 | 444 | 175 | * | 615 | 593 | 22 | | 0.89 |
| 29 | 1560002 | 633 | 515 | 118 | * | 621 | 621 | 0 | | 1.00 |
| 30 | 13934 | 636 | 359 | 277 | * | 618 | 618 | 0 | | 1.00 |
| 31 | 13459 | 577 | 510 | 67 | | 613 | 612 | 1 | * | 0.99 |
| 32 | 14066 | 577 | 436 | 141 | * | 619 | 619 | 0 | | 1.00 |
| 33 | 13675 | 631 | 513 | 118 | * | 620 | 620 | 0 | | 1.00 |
| 34 | 2973619 | 633 | 469 | 164 | * | 620 | 620 | 0 | | 1.00 |
| 35 | 13919 | 637 | 601 | 36 | * | 620 | 529 | 91 | | 0.28 |
| 36 | 2969221 | 629 | 437 | 192 | * | 615 | 615 | 0 | | 1.00 |
| 37 | 13372 | 577 | 402 | 175 | * | 621 | 621 | 0 | | 1.00 |
| 38 | 13420 | 580 | 509 | 71 | | 615 | 571 | 44 | * | 0.62 |
| 39 | 2809992 | 630 | 359 | 271 | * | 616 | 616 | 0 | | 1.00 |
| 40 | 13290 | 627 | 516 | 111 | * | 625 | 625 | 0 | | 1.00 |
| 41 | 1560003 | 628 | 387 | 241 | * | 605 | 605 | 0 | | 1.00 |
| 42 | 13336 | 592 | 449 | 143 | | 605 | 532 | 73 | * | 0.66 |
| 43 | 2783942 | 582 | 294 | 288 | * | 609 | 609 | 0 | | 1.00 |
| 44 | 4060301 | 581 | 376 | 205 | * | 623 | 563 | 60 | | 0.77 |
| 45 | 13637 | 579 | 398 | 181 | * | 609 | 545 | 64 | | 0.74 |
| 46 | 3200061 | 577 | 403 | 174 | | 606 | 432 | 174 | * | 0.50 |
| 47 | 13712 | 581 | 434 | 147 | * | 609 | 533 | 76 | | 0.66 |
| 48 | 2774403 | 578 | 238 | 340 | * | 601 | 601 | 0 | | 1.00 |
| 49 | 2737702 | 575 | 418 | 157 | * | 609 | 609 | 0 | | 1.00 |
| 50 | 2808359 | 579 | 374 | 205 | * | 609 | 593 | 16 | | 0.93 |
| 51 | 13421 | 587 | 182 | 405 | * | 580 | 580 | 0 | | 1.00 |
| 52 | 13600 | 577 | 174 | 403 | * | 579 | 579 | 0 | | 1.00 |
| 53 | 2775426 | 580 | 548 | 32 | | 577 | 445 | 132 | * | 0.20 |
| 54 | 13615 | 591 | 364 | 227 | * | 631 | 631 | 0 | | 1.00 |
| 55 | 13819 | 579 | 352 | 227 | | 618 | 551 | 67 | * | 0.77 |
| 56 | 2822009 | 616 | 255 | 361 | * | 641 | 641 | 0 | | 1.00 |
| 57 | 13835 | 631 | 419 | 212 | * | 622 | 604 | 18 | | 0.92 |
| 58 | 13836 | 576 | 171 | 405 | * | 632 | 370 | 262 | | 0.61 |
| 59 | 13822 | 642 | 301 | 341 | * | 638 | 638 | 0 | | 1.00 |
| 60 | 1450703 | 582 | 350 | 232 | * | 640 | 640 | 0 | | 1.00 |
| | Total: | 36163 | 25535 | 10628 | 41 | 36688 | 33795 | 2893 | 19 | 47.58 |
| | Average: | 602.7 | 425.6 | 177.1 | | 611.5 | 563.3 | 48.2 | | 0.79 |

The underlined numbers indicate all of the diet offered was consumed. 3.67 to 1 consumption ratio for Chow+PH Dental to Chow+PetLab Dental. 2.16 to 1 First Choice ratio for Chow+PH Dental to Chow+PetLab Dental.

TABLE 7
Day 4 Food Consumption and First Choice Data

| | | Grams of Diet Chow + | | | | Grams of Diet Chow + | | | | |
|---|---|---|---|---|---|---|---|---|---|---|
| | | PH Dental | | | | Pet Lab Dental | | | | |
| | | A | A | A | | B | B | B | | |
| | | Weigh | Weigh | Total | First | Weigh | Weigh | Total | First | Intake |
| | Dog | In | Out | Consumed | Choice | In | Out | Consumed | Choice | Ratio |
| 1 | 1484903 | 677 | 547 | 130 | * | 549 | 549 | 0 | | 1.00 |
| 2 | 13613 | 550 | 379 | 171 | * | 633 | 630 | 3 | | 0.98 |
| 3 | 2954062 | 553 | 382 | 171 | * | 592 | 581 | 11 | | 0.94 |
| 4 | 2814952 | 630 | 439 | 191 | * | 582 | 582 | 0 | | 1.00 |
| 5 | 2783526 | 559 | 482 | 77 | * | 600 | 509 | 91 | | 0.46 |
| 6 | 2794757 | 616 | 393 | 223 | * | 623 | 623 | 0 | | 1.00 |
| 7 | 2896151 | 546 | 411 | 135 | * | 626 | 626 | 0 | | 1.00 |
| 8 | 13592 | 608 | 337 | 271 | * | 625 | 587 | 38 | | 0.88 |
| 9 | 13530 | 602 | 332 | 270 | * | 620 | 602 | 18 | | 0.94 |
| 10 | 1484505 | 610 | 461 | 149 | | 619 | 540 | 79 | * | 0.65 |
| 11 | 13246 | 611 | 540 | 71 | * | 621 | 591 | 30 | | 0.70 |
| 12 | 2820669 | 599 | 414 | 185 | * | 585 | 547 | 38 | | 0.83 |
| 13 | 2813158 | 604 | 437 | 167 | * | 598 | 594 | 4 | | 0.98 |
| 14 | 13671 | 613 | 428 | 185 | * | 579 | 579 | 0 | | 1.00 |
| 15 | 2773709 | 605 | 477 | 128 | * | 577 | 549 | 28 | | 0.82 |
| 16 | 13883 | 629 | 455 | 174 | * | 583 | 582 | 1 | | 0.99 |
| 17 | 13941 | 624 | 498 | 126 | * | 636 | 636 | 0 | | 1.00 |
| 18 | 13916 | 625 | 435 | 190 | * | 578 | 578 | 0 | | 1.00 |
| 19 | 13522 | 622 | 491 | 131 | * | 623 | 623 | 0 | | 1.00 |
| 20 | 2780713 | 622 | 601 | 21 | | 623 | 455 | 168 | * | 0.11 |
| 21 | 13734 | 604 | 404 | 200 | * | 622 | 620 | 2 | | 0.99 |
| 22 | 13419 | 635 | 635 | 0 | * | 613 | 613 | 0 | | |
| 23 | 2772826 | 579 | 430 | 149 | | 619 | 613 | 6 | * | 0.96 |
| 24 | 1565206 | 620 | 620 | 0 | | 619 | 550 | 69 | * | 0.00 |
| 25 | 13657 | 576 | 440 | 136 | * | 615 | 605 | 10 | | 0.93 |
| 26 | 13922 | 622 | 544 | 78 | * | 630 | 630 | 0 | | 1.00 |
| 27 | 2754364 | 622 | 488 | 134 | * | 613 | 533 | 80 | | 0.63 |
| 28 | 2774209 | 620 | 501 | 119 | | 616 | 549 | 67 | * | 0.64 |
| 29 | 1560002 | 634 | 592 | 42 | * | 623 | 1623 | 0 | | 1.00 |
| 30 | 13934 | 636 | 381 | 255 | * | 617 | 617 | 0 | | 1.00 |
| 31 | 13459 | 577 | 488 | 89 | * | 613 | 613 | 0 | | 1.00 |
| 32 | 14066 | 577 | 434 | 143 | * | 619 | 619 | 0 | | 1.00 |
| 33 | 13675 | 630 | 508 | 122 | * | 621 | 621 | 0 | | 1.00 |
| 34 | 2973619 | 633 | 501 | 132 | * | 621 | 621 | 0 | | 1.00 |
| 35 | 13919 | 637 | 436 | 201 | * | 621 | 609 | 12 | | 0.94 |
| 36 | 2969221 | 630 | 449 | 181 | * | 615 | 615 | 0 | | 1.00 |
| 37 | 13372 | 578 | 420 | 158 | * | 622 | 622 | 0 | | 1.00 |
| 38 | 13420 | 580 | 466 | 114 | * | 615 | 615 | 0 | | 1.00 |
| 39 | 2809992 | 630 | 391 | 239 | * | 616 | 612 | 4 | | 0.98 |
| 40 | 13290 | 627 | 540 | 87 | * | 626 | 615 | 11 | | 0.89 |
| 41 | 1560003 | 629 | 326 | 303 | * | 603 | 603 | 0 | | 1.00 |
| 42 | 13336 | 593 | 392 | 201 | | 605 | 570 | 35 | * | 0.85 |
| 43 | 2783942 | 582 | 307 | 275 | * | 609 | 565 | 44 | | 0.86 |
| 44 | 4060301 | 581 | 377 | 204 | | 623 | 552 | 71 | * | 0.74 |
| 45 | 13637 | 579 | 243 | 336 | * | 609 | 609 | 0 | | 1.00 |
| 46 | 3200061 | 577 | 386 | 191 | * | 606 | 562 | 44 | | 0.81 |
| 47 | 13712 | 580 | 346 | 234 | * | 609 | 609 | 0 | | 1.00 |
| 48 | 2774403 | 577 | 349 | 228 | | 602 | 594 | 8 | * | 0.97 |
| 49 | 2737702 | 576 | 407 | 169 | * | 609 | 599 | 10 | | 0.94 |
| 50 | 2808359 | 580 | 380 | 200 | * | 608 | 572 | 36 | | 0.85 |
| 51 | 13421 | 587 | 182 | 405 | * | 577 | 577 | 0 | | 1.00 |
| 52 | 13600 | 577 | 173 | 404 | * | 578 | 459 | 119 | | 0.77 |
| 53 | 2775426 | 581 | 455 | 126 | * | 577 | 556 | 21 | | 0.86 |
| 54 | 13615 | 591 | 386 | 205 | * | 631 | 631 | 0 | | 1.00 |
| 55 | 13819 | 579 | 303 | 276 | * | 618 | 618 | 0 | | 1.00 |
| 56 | 2822009 | 616 | 314 | 302 | * | 641 | 640 | 1 | | 1.00 |
| 57 | 13835 | 632 | 449 | 183 | * | 622 | 615 | 7 | | 0.96 |
| 58 | 13836 | 577 | 385 | 192 | * | 633 | 618 | 15 | | 0.93 |
| 59 | 13822 | 642 | 315 | 327 | * | 638 | 638 | 0 | | 1.00 |
| 60 | 1450703 | 582 | 336 | 246 | * | 640 | 640 | 0 | | 1.00 |
| | Total: | 36170 | 25418 | 10752 | 52 | 36686 | 35505 | 1181 | 8 | 52.79 |
| | Average: | 602.8 | 423.6 | 179.2 | | 611.4 | 591.8 | 19.7 | | 10.89 |

TABLE 8
Day 5 Food Consumption and First Choice Data

| | | Grams of Diet Chow + | | | | Grams of Diet Chow + | | | | |
|---|---|---|---|---|---|---|---|---|---|---|
| | | PH Dental | | | | Pet Lab Dental | | | | |
| | | A | A | A | | B | B | B | | |
| | | Weigh | Weigh | Total | First | Weigh | Weigh | Total | First | Intake |
| | Dog | In | Out | Consumed | Choice | In | Out | Consumed | Choice | Ratio |
| 1 | 1484903 | 1677 | 546 | 131 | * | 549 | 549 | 0 | | 1.00 |
| 2 | 13613 | 550 | 447 | 103 | * | 633 | 594 | 39 | | 0.73 |
| 3 | 2954062 | 551 | 361 | 190 | * | 591 | 591 | 0 | | 1.00 |
| 4 | 2814952 | 631 | 395 | 236 | * | 583 | 583 | 0 | | 1.00 |
| 5 | 2783526 | 559 | 355 | 204 | * | 600 | 600 | 0 | | 1.00 |
| 6 | 2794757 | 616 | 397 | 219 | * | 623 | 623 | 0 | | 1.00 |
| 7 | 2896151 | 547 | 371 | 176 | * | 625 | 625 | 0 | | 1.00 |
| 8 | 13592 | 609 | 515 | 94 | * | 624 | 535 | 89 | | 0.51 |
| 9 | 13530 | 602 | 149 | 111 | | 620 | 439 | 181 | * | 0.38 |
| 10 | 1484505 | 610 | 399 | 211 | * | 620 | 539 | 81 | | 0.72 |
| 11 | 13246 | 610 | 519 | 91 | * | 622 | 561 | 61 | | 0.60 |
| 12 | 2820669 | 601 | 390 | 211 | | 595 | 570 | 25 | * | 0.89 |
| 13 | 2813158 | 604 | 493 | 111 | * | 598 | 525 | 73 | | 0.60 |
| 14 | 13671 | 612 | 442 | 170 | * | 580 | 559 | 21 | | 0.89 |
| 15 | 2773709 | 604 | 418 | 186 | * | 578 | 578 | 0 | | 1.00 |
| 16 | 13883 | 628 | 431 | 197 | * | 583 | 583 | 0 | | 1.00 |
| 17 | 13941 | 624 | 483 | 141 | * | 636 | 633 | 3 | | 0.98 |
| 18 | 13916 | 625 | 404 | 221 | * | 577 | 577 | 0 | | 1.00 |
| 19 | 13522 | 624 | 624 | 0 | | 623 | 492 | 131 | * | 0.00 |
| 20 | 2780713 | 622 | 217 | 405 | * | 623 | 623 | 0 | | 1.00 |
| 21 | 13734 | 604 | 434 | 170 | * | 622 | 622 | 0 | | 1.00 |
| 22 | 13419 | 636 | 485 | 151 | * | 613 | 613 | 0 | | 1.00 |
| 23 | 2772826 | 579 | 1421 | 158 | * | 620 | 610 | 10 | | 0.94 |
| 24 | 1565206 | 622 | 530 | 92 | | 619 | 585 | 34 | * | 0.73 |
| 25 | 13657 | 577 | 357 | 220 | * | 617 | 606 | 11 | | 0.95 |
| 26 | 13922 | 622 | 525 | 97 | * | 630 | 630 | 0 | | 1.00 |
| 27 | 2754364 | 624 | 483 | 141 | * | 614 | 576 | 38 | | 10.79 |
| 28 | 2774209 | 621 | 491 | 130 | | 616 | 536 | 80 | * | 0.62 |
| 29 | 1560002 | 634 | 604 | 30 | | 624 | 624 | 0 | * | 1.00 |
| 30 | 13934 | 637 | 417 | 220 | * | 618 | 618 | 0 | | 1.00 |
| 31 | 13459 | 578 | 501 | 77 | * | 613 | 595 | 18 | | 0.81 |
| 32 | 14066 | 578 | 441 | 137 | * | 621 | 617 | 4 | | 0.97 |
| 33 | 13675 | 630 | 501 | 129 | * | 622 | 622 | 0 | | 1.00 |
| 34 | 2973619 | 634 | 491 | 143 | * | 621 | 621 | 0 | | 1.00 |
| 35 | 13919 | 638 | 524 | 114 | * | 621 | 460 | 161 | | 0.41 |
| 36 | 2969221 | 629 | 428 | 201 | * | 616 | 616 | 0 | | 1.00 |
| 37 | 13372 | 578 | 383 | 195 | * | 622 | 622 | 0 | | 1.00 |
| 38 | 13420 | 581 | 496 | 85 | | 616 | 589 | 27 | * | 0.76 |
| 39 | 2809992 | 630 | 480 | 150 | | 616 | 549 | 67 | * | 0.69 |
| 40 | 13290 | 628 | 585 | 43 | * | 626 | 626 | 0 | | 1.00 |
| 41 | 1560003 | 628 | 344 | 284 | * | 605 | 605 | 0 | | 1.00 |
| 42 | 13336 | 591 | 399 | 192 | * | 605 | 594 | 11 | | 0.95 |
| 43 | 2783942 | 581 | 240 | 341 | * | 610 | 610 | 0 | | 1.00 |
| 44 | 4060301 | 582 | 416 | 166 | | 624 | 584 | 40 | * | 0.81 |
| 45 | 13637 | 578 | 397 | 181 | | 610 | 573 | 37 | * | 0.83 |
| 46 | 3200061 | 577 | 286 | 291 | * | 607 | 605 | 2 | | 0.99 |
| 47 | 13712 | 579 | 446 | 133 | | 609 | 588 | 21 | * | 0.86 |
| 48 | 2774403 | 575 | 369 | 206 | | 602 | 581 | 21 | * | 0.91 |
| 49 | 2737702 | 573 | 462 | 111 | | 610 | 553 | 57 | * | 0.66 |
| 50 | 2808359 | 579 | 382 | 197 | * | 609 | 542 | 67 | | 10.75 |
| 51 | 13421 | 586 | 193 | 393 | * | 581 | 581 | 0 | | 1.00 |
| 52 | 13600 | 576 | 176 | 400 | * | 581 | 581 | 0 | | 1.00 |
| 53 | 2775426 | 579 | 502 | 77 | * | 577 | 499 | 78 | | 0.50 |
| 54 | 13615 | 590 | 349 | 241 | * | 631 | 629 | 2 | | 0.99 |
| 55 | 13819 | 577 | 382 | 195 | | 617 | 614 | 3 | * | 0.98 |
| 56 | 2822009 | 616 | 244 | 372 | * | 641 | 641 | 0 | | 1.00 |
| 57 | 13835 | 629 | 461 | 168 | * | 622 | 600 | 22 | | 0.88 |
| 58 | 13836 | 575 | 495 | 80 | * | 632 | 305 | 327 | | 0.20 |
| 59 | 13822 | 640 | 404 | 236 | * | 640 | 640 | 10 | | 1.00 |
| 60 | 1450703 | 581 | 353 | 228 | * | 641 | 641 | 0 | | 1.00 |
| | Total: | 36158 | 25575 | 10583 | 46 | 36724 | 34882 | 1842 | 14 | 51.29 |
| | Average: | 602.6 | 426.3 | 176.4 | | 612.1 | 581.4 | 30.7 | | 0.85 |

The underlined numbers indicate all of the diet offered was consumed. 5.75 to 1 consumption ratio for Chow+PH Dental to Chow+PetLab Dental. 3.29 to 1 First Choice ratio for Chow+PH Dental to Chow+PetLab Dental.

TABLE 9
Summary Food Consumption and First Choice Data

| | | Grams of Diet Chow + PH Dental | | | | Grams of Diet Chow + Pet Lab Dental | | | | |
|---|---|---|---|---|---|---|---|---|---|---|
| | | A | A | A | | B | B | B | | |
| | | Weigh | Weigh | Total | First | Weigh | Weigh | Total | First | Intake |
| | Dog | In | Out | Consumed | Choice | In | Out | Consumed | Choice | Ratio |
| 1 | 1484903 | 3385 | 2627 | 758 | 4 | 2745 | 2743 | 2 | 1 | 1.00 |
| 2 | 13613 | 2751 | 2096 | 655 | 4 | 3167 | 3034 | 133 | 1 | 0.83 |
| 3 | 2954062 | 2762 | 1931 | 831 | 4 | 2959 | 2872 | 87 | 1 | 0.91 |
| 4 | 2814952 | 3151 | 2117 | 1034 | 5 | 2913 | 2886 | 27 | 0 | 0.97 |
| 5 | 2783526 | 2794 | 2117 | 677 | 3 | 3000 | 2812 | 188 | 2 | 0.78 |
| 6 | 2794757 | 3077 | 2006 | 1071 | 5 | 3114 | 3079 | 35 | 0 | 0.97 |
| 7 | 2896151 | 2731 | 1766 | 965 | 4 | 3130 | 2931 | 199 | 1 | 0.83 |
| 8 | 13592 | 3038 | 2200 | 838 | 5 | 3125 | 2953 | 172 | 0 | 0.83 |
| 9 | 13530 | 3010 | 2282 | 728 | 2 | 3098 | 2396 | 702 | 3 | 0.51 |
| 10 | 1484505 | 3051 | 2192 | 859 | 4 | 3096 | 2748 | 348 | 1 | 0.71 |
| 11 | 13246 | 3053 | 2768 | 285 | 4 | 3106 | 2784 | 322 | 1 | 0.47 |
| 12 | 2820669 | 2998 | 2314 | 684 | 2 | 2935 | 2676 | 259 | 3 | 0.73 |
| 13 | 2813158 | 3020 | 2486 | 534 | 4 | 2988 | 2522 | 466 | 1 | 0.53 |
| 14 | 13671 | 3057 | 2349 | 708 | 4 | 2896 | 2823 | 73 | 1 | 0.91 |
| 15 | 2773709 | 3024 | 2405 | 619 | 5 | 2887 | 2723 | 164 | 0 | 0.79 |
| 16 | 13883 | 3142 | 2454 | 688 | 5 | 2913 | 2892 | 21 | 0 | 0.97 |
| 17 | 13941 | 3118 | 2490 | 628 | 4 | 3180 | 3139 | 41 | 1 | 0.94 |
| 18 | 13916 | 3126 | 2096 | 1030 | 4 | 2885 | 2874 | 11 | 1 | 0.99 |
| 19 | 13522 | 3116 | 2833 | 283 | 2 | 3116 | 2877 | 239 | 3 | 0.54 |
| 20 | 2780713 | 3110 | 1945 | 1165 | 3 | 3115 | 2708 | 407 | 2 | 0.74 |
| 21 | 13734 | 3016 | 2204 | 812 | 4 | 3110 | 2949 | 161 | 1 | 0.83 |
| 22 | 13419 | 3179 | 2782 | 397 | 4 | 3064 | 2597 | 467 | 1 | 0.46 |
| 23 | 2772826 | 2894 | 2016 | 878 | 3 | 3101 | 2968 | 133 | 2 | 0.87 |
| 24 | 1565206 | 3105 | 2799 | 306 | 1 | 3094 | 2781 | 313 | 4 | 0.49 |
| 25 | 13657 | 2883 | 2259 | 624 | 5 | 3083 | 3062 | 21 | 0 | 0.97 |
| 26 | 13922 | 3109 | 2460 | 649 | 2 | 3150 | 3027 | 123 | 3 | 0.84 |
| 27 | 2754364 | 3114 | 2464 | 650 | 2 | 3069 | 2609 | 460 | 3 | 0.59 |
| 28 | 2774209 | 3100 | 2320 | 780 | 1 | 3081 | 2740 | 341 | 4 | 0.70 |
| 29 | 1560002 | 3168 | 2672 | 496 | 4 | 3114 | 3108 | 6 | 1 | 0.99 |
| 30 | 13934 | 3184 | 1622 | 1562 | 5 | 3089 | 3076 | 13 | 0 | 0.99 |
| 31 | 13459 | 2887 | 2475 | 412 | 2 | 3066 | 2959 | 107 | 3 | 0.79 |
| 32 | 14066 | 2885 | 2192 | 693 | 5 | 3099 | 3079 | 20 | 0 | 0.97 |
| 33 | 13675 | 3152 | 2570 | 582 | 4 | 3107 | 3016 | 91 | 1 | 0.86 |
| 34 | 2973619 | 3168 | 2515 | 653 | 4 | 3106 | 2981 | 125 | 1 | 0.84 |
| 35 | 13919 | 3188 | 2574 | 614 | 5 | 3102 | 2647 | 455 | 0 | 0.57 |
| 36 | 2969221 | 3147 | 2086 | 1061 | 5 | 3077 | 3077 | 0 | 0 | 1.00 |
| 37 | 13372 | 2890 | 2048 | 842 | 5 | 3112 | 3088 | 24 | 0 | 0.97 |
| 38 | 13420 | 2903 | 2430 | 473 | 3 | 3078 | 2942 | 136 | 2 | 0.78 |
| 39 | 2809992 | 3150 | 1862 | 1288 | 4 | 3079 | 3007 | 72 | 1 | 0.95 |
| 40 | 13290 | 3138 | 2684 | 454 | 4 | 3129 | 3098 | 31 | 1 | 0.94 |
| 41 | 1560003 | 3142 | 2314 | 828 | 3 | 3020 | 2667 | 353 | 2 | 0.70 |
| 42 | 13336 | 2962 | 2163 | 799 | 2 | 3025 | 2855 | 170 | 3 | 0.82 |
| 43 | 2783942 | 2909 | 1676 | 1233 | 5 | 3045 | 2908 | 137 | 0 | 0.90 |
| 44 | 4060301 | 2907 | 1925 | 982 | 2 | 3116 | 2932 | 184 | 3 | 0.84 |
| 45 | 13637 | 2896 | 1805 | 1091 | 3 | 3046 | 2887 | 159 | 2 | 0.87 |
| 46 | 3200061 | 2886 | 1693 | 1193 | 4 | 3031 | 2716 | 315 | 1 | 0.79 |
| 47 | 13712 | 2900 | 2205 | 695 | 3 | 3045 | 2792 | 253 | 2 | 0.73 |
| 48 | 2774403 | 2885 | 1590 | 1295 | 2 | 3007 | 2977 | 30 | 3 | 0.98 |
| 49 | 2737702 | 2874 | 2233 | 641 | 4 | 3046 | 2882 | 164 | 1 | 0.80 |
| 50 | 2808359 | 2898 | 1994 | 904 | 4 | 3043 | 2882 | 161 | 1 | 0.85 |
| 51 | 13421 | 2933 | 1078 | 18555 | 5 | 2897 | 2873 | 24 | 0 | 0.99 |
| 52 | 13600 | 2883 | 872 | 20115 | 5 | 2899 | 2778 | 121 | 0 | 0.94 |
| 53 | 2775426 | 2899 | 2623 | 276 | 3 | 2883 | 2542 | 341 | 2 | 0.45 |
| 54 | 13615 | 2954 | 1954 | 10005 | 5 | 3155 | 3152 | 3 | 0 | 1.00 |
| 55 | 13819 | 2894 | 1695 | 11992 | 2 | 3087 | 2972 | 115 | 3 | 0.91 |
| 56 | 2822009 | 3081 | 1627 | 14543 | 3 | 3206 | 3109 | 97 | 2 | 0.94 |
| 57 | 13835 | 3156 | 2282 | 874 | 4 | 3108 | 2969 | 139 | 1 | 0.86 |
| 58 | 13836 | 2880 | 1956 | 924 | 4 | 3161 | 2479 | 682 | 1 | 0.58 |
| 59 | 13822 | 3206 | 1803 | 1403 | 5 | 3194 | 3194 | 0 | 0 | 1.00 |
| 60 | 1450703 | 2910 | 1884 | 10265 | 5 | 3202 | 3202 | 0 | 0 | 1.00 |
| | Total: | 180829 | 129880 | 50949 | 222 | 183494 | 173051 | 10443 | 78 | 49.30 |
| | Average: | 3013.8 | 2164.7 | 849.2 | | 3058.2 | 2884.2 | 174.1 | | 0.82 |

$$4.88 to 1 consumption ratio for Chow + P H Dental to Chow + PetLab Dental .$$

$$2.85 to 1 First Choice ratio for Chow + P H Dental to Chow + PetLab Dental .$$

### Example 2: Canine Preference Results: Pet Honesty Fresh Breath Dental Powder Composition (PH Dental) vs PlaqueOff Powder (PlaqueOff)

### Consumption Summary

The total amount of food consumed by the 20 dogs during the 2 days of this test was 8,946 Grams as follows: Diet A as Chow+PH Dental: 6,028 Grams or 67.4%; Diet B as Chow+PlaqueOff: 2,918 Grams or 32.6%. PH Dental composition includes Brown Algae (Ascophyllum nodosum), Sodium Hexametaphosphate, Fresh Breath Blend (Decaffeinated Green Tea & Parsley), Postbiotic (L. plantarum) and Inactive ingredients Brown Rice Flour, Pork Liver, Bentonite, Natural Mint Flavor, Mixed Tocopherols. PlaqueOff powder includes Kelp, Natural iodine 0.08% Crude protein 5-8% Crude fat 2-4%, and Crude ash 15-27% Crude fibre<8%. There is a 2.07 to 1 consumption ratio of Chow+PH Dental to Chow+PlaqueOff.

Using a Wilcoxon Signed Rank test there is significant reason to show and support a consumption difference between the two Diets (P=0.0006). A 2-way analysis of variance (2-Way ANOVA) indicates that there is significant reason to suspect a difference in consumption of the 2 diets when the variance in consumption of the dogs is accounted for. (F=22.696, P=0.0001)

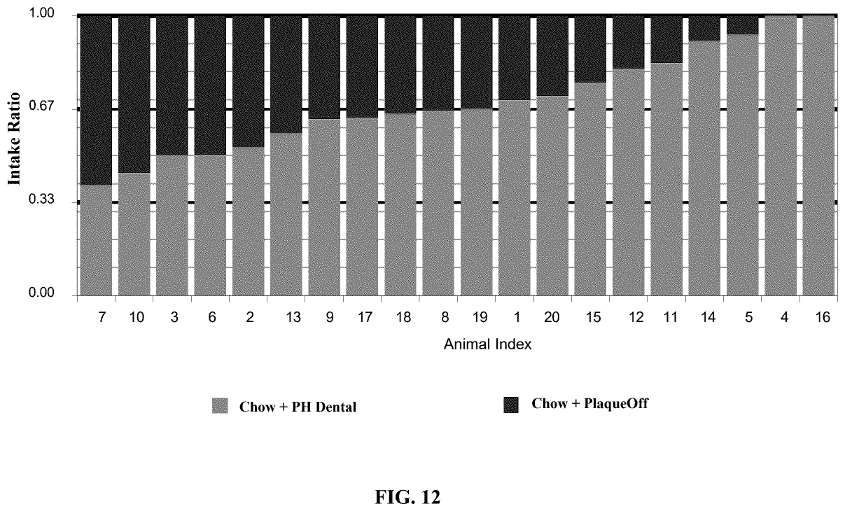

Intake Ratio Chart is shown in FIG. 12. The individual Intake Ratios for each of the 20 dogs are sorted in ascending order for Chow+PH Dental.

The average of the 20 individual Intake Ratios for each Diet are as follows: Diet A as Chow+PH Dental:Average IR=0.693. Diet B as Chow+PlaqueOff: Average IR=0.307.

### Preference By First Choice

Over both days Chow+PH Dental was chosen first on 31 occasions and Chow+PlaqueOff was chosen first on 9 occasions. A significant First Choice is detected and tends to favor Chow+PH Dental. The Chi² probability is p=0.0005

Over both days 14 dogs chose Chow+PH Dental first every day and 3 dogs chose Chow+PlaqueOff first every day, the other 3 were undecided. There is significant reason to suspect that this result is not random as a definite preference is detected. The Chi² probability is p=0.0000.

### Daily Consumption Summary

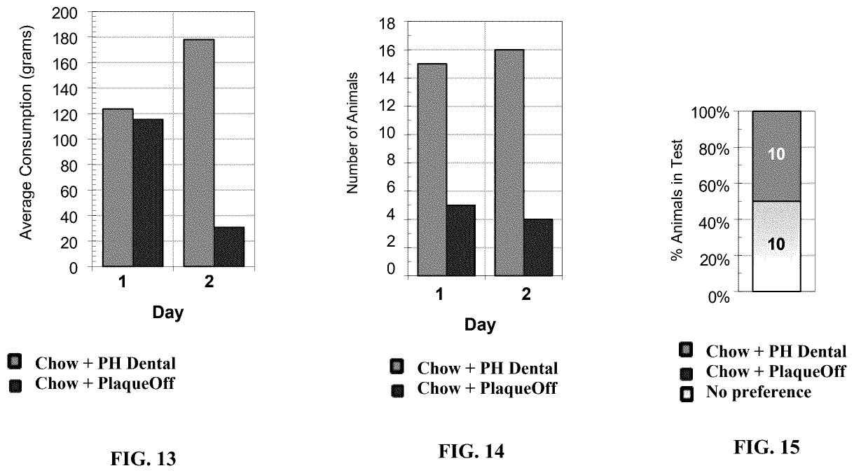

The average daily consumption of each diet is shown in FIG. 13 and listed below for both days. The consumption of each diet is compared using a separate paired t-Test within each day (20 dogs by 2 diets) to assess significance in consumption difference for that day.

Average Daily Consumption Data

| Day | Diet A | Diet B | Total | P of t | Significant |
|---|---|---|---|---|---|
| 1 | 123.6 | 115.3 | 238.8 | 0.7907 | NO |
| 2 | 177.9 | 30.7 | 208.5 | 0.0000 | YES! |

### Daily Preference By First Choice

The diet of First Choice preference is scored daily for each dog and the counts are shown in FIG. 14 and listed below. A Chi² test compares the daily counts for Chow+PH Dental and Chow+PlaqueOff to the counts that might have been expected if there was no First-Choice difference in the two diets. In this case that would be a 10:10 ratio or both diets chosen equally. Now, a Significant result (YES!) here indicates that these products were not chosen equally. Whereas, a non-Significant result (NO) means that there is no evidence to suspect a First Choice difference.

Daily First Choice Counts

| Day | Diet A | Diet B | P of Chi² | Significant |
|---|---|---|---|---|
| 1 | 15 | 5 | 0.0253 | YES! |
| 2 | 16 | 4 | 0.0073 | YES! |

### Preference Scoring by Intake Ratio

It is a popular contention that an animal shows a clear preference by consuming twice as much of one diet as the other. This is measured as a 2:1 consumption ratio or a 0.6667 Intake Ratio.

Scoring is achieved by reviewing the average intake ratios for each dog in the test and scoring one point for the Diet with an Intake Ratio greater than or equal to 0.6667.

As shown in FIG. 15, using the 2:1 consumption criteria in the Intake Preference chart to the right, 10 dogs prefer Chow+PH Dental, no dogs prefer Chow+PlaqueOff and the other 10 dogs show no Preference.

### Methods and Procedures

The kennel facility is registered with the USDA No. 23-R-0126 under the Animal Welfare Act. The kennel had a 12-hour-light/12-hour-dark cycle. Every attempt was made to keep temperature ranges within targeted conditions (from 50° to 80° F.) in accordance with the Animal Welfare Act. Cages and feed bowls were cleaned daily and sanitized in accordance with the Animal Welfare Act.

The purpose of the study was to assess the palatability of Chow+PH Dental and Chow+PlaqueOff. On Nov. 4, 2024, 2 containers of test article PH Dental and 2 containers of test article PlaqueOff were received.

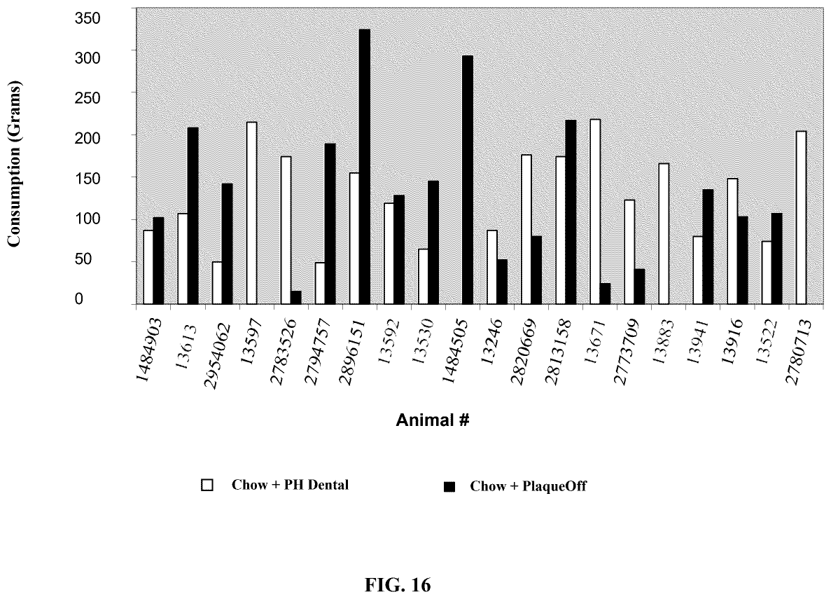

FIG. 16 and Table 10 shows the Day 1 food consumption and first choice data.

TABLE 10
Day 1 Food Consumption and First Choice Data

| | | Grams of Diet Chow + PH Dental | | | | Grams of Diet Chow + PlaqueOff | | | | |
|---|---|---|---|---|---|---|---|---|---|---|
| | | A | A | A | | B | B | B | | |
| | | Weigh | Weigh | Total | First | Weigh | Weigh | Total | First | Intake |
| | Dog | In | Out | Consumed | Choice | In | Out | Consumed | Choice | Ratio |
| 1 | 1484903 | 677 | 590 | 87 | * | 549 | 447 | 102 | | 0.46 |
| 2 | 13613 | 549 | 442 | 107 | * | 634 | 426 | 208 | | 0.34 |
| 3 | 2954062 | 553 | 503 | 50 | * | 592 | 450 | 142 | | 0.26 |
| 4 | 13597 | 630 | 415 | 215 | * | 582 | 582 | 0 | | 1.00 |
| 5 | 2783526 | 558 | 384 | 174 | | 600 | 585 | 15 | * | 0.92 |
| 6 | 2794757 | 615 | 566 | 49 | * | 624 | 435 | 189 | | 0.21 |
| 7 | 2896151 | 546 | 391 | 155 | | 626 | 302 | 324 | * | 0.32 |
| 8 | 13592 | 608 | 489 | 119 | * | 625 | 497 | 128 | | 0.48 |
| 9 | 13530 | 601 | 536 | 65 | | 620 | 475 | 145 | * | 0.31 |
| 10 | 1484505 | 610 | 610 | 0 | | 619 | 326 | 293 | * | 0.00 |
| 11 | 13246 | 612 | 525 | 87 | * | 621 | 569 | 52 | | 0.63 |
| 12 | 2820669 | 600 | 424 | 176 | * | 585 | 505 | 80 | | 0.69 |
| 13 | 2813158 | 604 | 430 | 174 | * | 598 | 381 | 217 | | 0.45 |
| 14 | 13671 | 611 | 393 | 218 | * | 579 | 555 | 24 | | 0.90 |
| 15 | 2773709 | 605 | 482 | 123 | * | 579 | 538 | 41 | | 0.75 |
| 16 | 13883 | 630 | 464 | 166 | * | 584 | 584 | 0 | | 1.00 |
| 17 | 13941 | 623 | 543 | 80 | * | 637 | 502 | 135 | | 0.37 |
| 18 | 13916 | 625 | 477 | 148 | * | 578 | 475 | 103 | | 0.59 |
| 19 | 13522 | 623 | 549 | 74 | | 624 | 517 | 107 | * | 0.41 |
| 20 | 2780713 | 623 | 419 | 204 | * | 623 | 623 | 0 | | 1.00 |
| | Total: | 12103 | 9632 | 2471 | 15 | 12079 | 9774 | 2305 | 5 | 11.08 |
| | Average: | 605.2 | 481.6 | 123.6 | | 604.0 | 488.7 | 115.3 | | 10.55 |

$$1.07 to 1 consumption ratio for Chow + P H Dental to Chow + PlaqueOff .$$

$$3. to 1 First Choice ratio for Chow + P H Dental to Chow + PlaqueOff$$

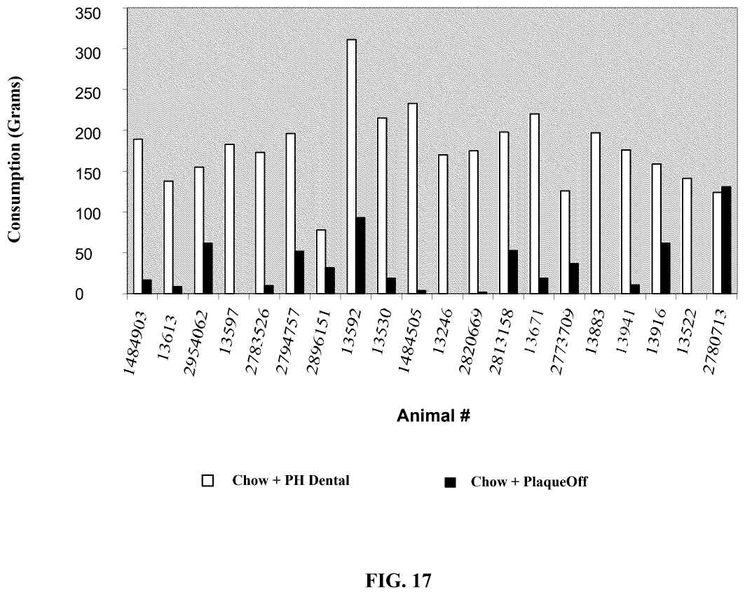

Twenty male and female Beagles identified by ear tattoo and cage number were presented the test diets on an individual basis. Two stainless steel bowls, each containing approximately 405 grams of diet, were offered once daily for 2 days. To create each diet, 5 grams of the designated powder were mixed into 400 grams of Purina Dog Chow. Bowl placement was reversed daily and both bowls were presented for 30 minutes. If one diet was completely consumed prior to the end of the 30 minutes, both bowls were removed. Food consumption and first choice preference were recorded for each dog. FIG. 17 and Table 11 shows the Day 2 food consumption and first choice data.

TABLE 11
Day 2 Food Consumption and First Choice Data

| | | Grams of Diet Chow + | | | | Grams of Diet Chow + | | | | |
|---|---|---|---|---|---|---|---|---|---|---|
| | | PH Dental | | | | PlaqueOff | | | | |
| | | A | A | | | B | B | | | |
| | | Weigh | Weigh | A | First | Weigh | Weigh | B | First | |
| | Dog | In | Out | Total | Choice | In | Out | Total | Choice | IR |
| 1 | 1484903 | 677 | 488 | 189 | * | 550 | 533 | 17 | | 0.92 |
| 2 | 13613 | 549 | 411 | 138 | * | 634 | 625 | 9 | | 0.94 |
| 3 | 2954062 | 552 | 397 | 155 | * | 592 | 530 | 62 | | 0.71 |
| 4 | 13597 | 630 | 447 | 183 | * | 583 | 583 | 0 | | 1.00 |
| 5 | 2783526 | 558 | 385 | 173 | | 601 | 591 | 10 | * | 0.95 |
| 6 | 2794757 | 615 | 419 | 196 | * | 623 | 571 | 52 | | 0.79 |
| 7 | 2896151 | 546 | 468 | 78 | * | 627 | 595 | 32 | | 0.71 |
| 8 | 13592 | 608 | 297 | 311 | * | 625 | 532 | 93 | | 0.77 |
| 9 | 13530 | 602 | 387 | 215 | | 620 | 601 | 19 | * | 0.92 |
| 10 | 1484505 | 610 | 377 | 233 | | 619 | 615 | 4 | * | 0.98 |
| 11 | 13246 | 611 | 441 | 170 | * | 621 | 621 | 0 | | 1.00 |
| 12 | 2820669 | 600 | 425 | 175 | * | 585 | 583 | 2 | | 0.99 |
| 13 | 2813158 | 604 | 406 | 198 | | 598 | 545 | 53 | * | 0.79 |
| 14 | 13671 | 611 | 391 | 220 | * | 579 | 560 | 19 | | 0.92 |
| 15 | 2773709 | 605 | 479 | 126 | * | 578 | 541 | 37 | | 0.77 |
| 16 | 13883 | 628 | 431 | 197 | * | 583 | 583 | 0 | | 1.00 |
| 17 | 13941 | 625 | 449 | 176 | * | 636 | 625 | 11 | | 0.94 |
| 18 | 13916 | 625 | 466 | 159 | * | 577 | 515 | 62 | | 0.72 |
| 19 | 13522 | 624 | 483 | 141 | * | 624 | 624 | 0 | | 1.00 |
| 20 | 2780713 | 622 | 498 | 124 | * | 623 | 492 | 131 | | 0.49 |
| | Total: | 12102 | 8545 | 3557 | 16 | 12078 | 11465 | 613 | 4 | 17.30 |
| | Average: | 605.1 | 427.3 | 177.9 | | 603.9 | 573.3 | 30.7 | | 0.87 |

$$5.8 to 1 consumption ratio for Chow + P H Dental to Chow + PlaqueOff .$$

$$4. to 1 First Choice ratio for Chow + P H Dental to Chow + PlaqueOff .$$

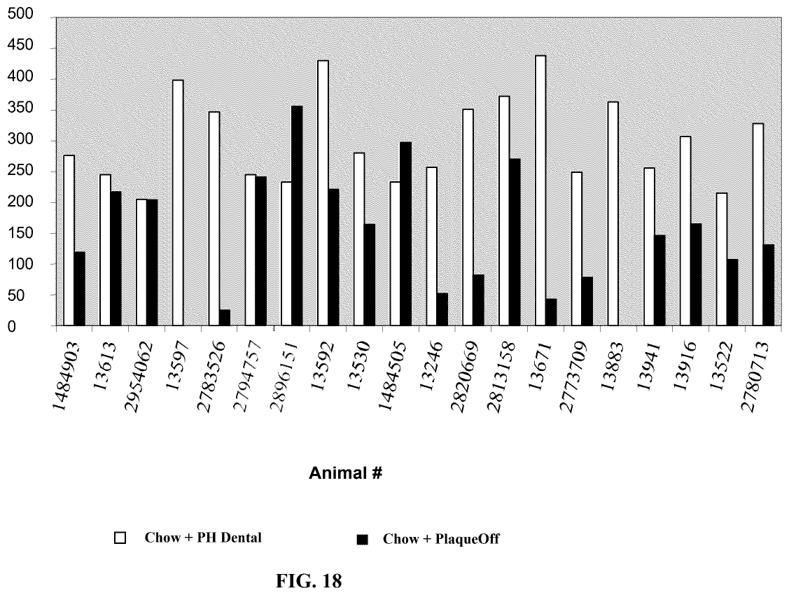

FIG. 18 and Table 12 shows the total food consumption and first choice data.

Summary Table 12
Total Food Consumption and First Choice Data

| | | Grams of Diet Chow + | | | | Grams of Diet Chow + | | | | |
|---|---|---|---|---|---|---|---|---|---|---|
| | | PH Dental | | | | PlaqueOff | | | | |
| | | A | A | A | | B | B | B | | |
| | | Weigh | Weigh | Total | First | Weigh | Weigh | Total | First | Intake |
| | Dog | In | Out | Consumed | Choice | In | Out | Consumed | Choice | Ratio |
| 1 | 1484903 | 1354 | 1078 | 276 | 2 | 1099 | 980 | 119 | 0 | 0.70 |
| 2 | 13613 | 1098 | 853 | 245 | 2 | 1268 | 1051 | 217 | 0 | 0.53 |
| 3 | 2954062 | 1105 | 900 | 205 | 2 | 1184 | 980 | 204 | 0 | 0.50 |
| 4 | 13597 | 1260 | 862 | 398 | 2 | 1165 | 1165 | 0 | 0 | 1.00 |
| 5 | 2783526 | 1116 | 769 | 347 | 0 | 1201 | 1176 | 25 | 2 | 0.93 |
| 6 | 2794757 | 1230 | 985 | 245 | 2 | 1247 | 1006 | 241 | 0 | 0.50 |
| 7 | 2896151 | 1092 | 859 | 233 | 1 | 1253 | 897 | 356 | 1 | 0.40 |
| 8 | 13592 | 1216 | 786 | 430 | 2 | 1250 | 1029 | 221 | 0 | 0.66 |
| 9 | 13530 | 1203 | 923 | 280 | 0 | 1240 | 1076 | 164 | 2 | 0.63 |
| 10 | 1484505 | 1220 | 987 | 233 | 0 | 1238 | 941 | 297 | 2 | 0.44 |
| 11 | 13246 | 1223 | 966 | 257 | 2 | 1242 | 1190 | 52 | 0 | 0.83 |
| 12 | 2820669 | 1200 | 849 | 351 | 2 | 1170 | 1088 | 82 | 0 | 0.81 |
| 13 | 2813158 | 1208 | 836 | 372 | 1 | 1196 | 926 | 270 | 1 | 0.58 |
| 14 | 13671 | 1222 | 784 | 438 | 2 | 1158 | 1115 | 43 | 0 | 0.91 |
| 15 | 2773709 | 1210 | 961 | 249 | 2 | 1157 | 1079 | 78 | 0 | 0.76 |
| 16 | 13883 | 1258 | 895 | 363 | 2 | 1167 | 1167 | 0 | 0 | 1.00 |
| 17 | 13941 | 1248 | 992 | 256 | 2 | 1273 | 1127 | 146 | 0 | 0.64 |
| 18 | 13916 | 1250 | 943 | 307 | 2 | 1155 | 990 | 165 | 0 | 0.65 |
| 19 | 13522 | 1247 | 1032 | 215 | 1 | 1248 | 1141 | 107 | 1 | 0.67 |
| 20 | 2780713 | 1245 | 917 | 328 | 2 | 1246 | 1115 | 131 | 0 | 0.71 |
| | Total: | 24205 | 18177 | 6028 | 31 | 24157 | 21239 | 2918 | 9 | 13.86 |
| | Average: | 1210.3 | 908.9 | 301.4 | | 1207.9 | 1062.0 | 145.9 | | 0.69 |

$$2.07 to 1 consumption ratio for Chow + P H Dental to Chow + PlaqueOff .$$

$$3.44 to 1. First Choice ratio for Chow + P H Dental to Chow + PlaqueOff .$$

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method of improving dental health of a pet or animal, comprising:
a. Administering a dental powder to the pet or animal over a time period, wherein the dental powder comprises seaweed, a tartar control agent, a botanical blend, and a postbiotic;
b. Improving dental health or oral health of the pet or animal by administering the dental powder on a food product daily for at least once a week.

2. The method of claim 1, wherein administering the dental powder on a food product ensures compliance by the pet or animal to contact the dental powder with the surfaces of the oral cavity for a period of time.

3. The method of claim 1, wherein the postbiotic is Lactobacillus plantarum and L. plantarum inhibits the growth of harmful bacteria in the mouth and reduces dental plaque, dental tartar, tooth discoloration, and breath malodor over the treatment period of at least one week.

4. The method of claim 1, wherein dental health or oral health includes breath odor, tooth whiteness, plaque and tartar, gum health, overall dental health related to oral comfort including oral hydration, gum soothing, decreasing oral microbe growth and inhibiting of oral microbiofilms, or any combination thereof.

5. The method of claim 1, further comprising increasing palatability of a food product and contacting the dental powder composition with surfaces of the oral cavity for a feeding period.

6. The method of claim 1, wherein applying the dental powder to pet food comprises administering the dental powder in the amount of at least 2.5 grams per food serving.

7. The method of claim 1, wherein administering the dental powder on the food product has a preference over other dental powders.

8. The method of claim 1, wherein the anti-tartar agent comprises sodium hexametaphosphate.

9. The method of claim 1, wherein the seaweed is selected from the group consisting of: kelp or sea vegetables (algae) include green, brown, red, and blue-green algae, ascophyllum nodosum, and derivatives of algae.

10. A dental powder composition for treating the dental health or oral health of a pet or animal; comprising: effective amount of seaweed, anti-tartar agent, a botanical blend, and a postbiotic, a probiotic, or a prebiotic.

11. The dental powder composition of claim 10, wherein the anti-tartar agent comprises sodium hexametaphosphate.

12. The dental powder composition of claim 10, wherein the seaweed, sodium hexametaphosphate, a botanical blend, a postbiotic and/or parts thereof may be mixed, combined, disposed, suspended, or otherwise contacted with an orally acceptable vehicle.

13. The dental powder composition of claim 10, wherein the postbiotic is Lactobacillus plantarum to improve oral health by inhibiting the growth of harmful bacteria and reducing cavities.

14. The dental powder composition of claim 10, wherein the postbiotic, prebiotic, or probiotic is selected from one or more of the genera selected from: Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus and Weissella. In an embodiment, the lactic acid bacteria is selected from one or more or all of: Lactobacillus plantarum, Leuconostoc mesenteroides, Lactobacillus rhamnosus, Lactobacillus pentosus, Lactobacillus brevis, Lactococus lactis, Lactobacillus acidophilus, Lactobacillus brevis, Lactobacillus casei, Lactobacillus delbrueckii, Lactobacillus fermenturn, Lactobacillus gasseri, Lactobacillus johnsonii, Lactobacillus lactis, Lactobacillus paracasei, Lactobacillus reuteri, Pediococcus pentosaceus and Pedicoccus acidilacti.

15. The dental powder composition of claim 10, wherein the amount of the probiotic, prebiotic, or postbiotic present in the dental powder product or the dental powder composition thereof may be from about 0.001 weight percent to about 3 weight percent, based on a dried, rehydrated, or fresh weight of the probiotic, prebiotic, and/or postbiotic relative to a total weight of the dental powder product or the dental powder composition thereof.

16. The dental powder composition of claim 10, wherein the seaweed is selected from the group consisting of: kelp or sea vegetables (also known as algae) include green, brown, red, and blue-green algae, derivatives of algae.

17. The dental powder composition of claim 10, wherein the amount of the seaweed, kelp, or sea vegetable present in the dental powder product or the dental powder composition thereof may be from about 1.0 weight percent to about 30 weight percent, based on a dried, rehydrated, or fresh weight of the dental powder composition thereof.

18. The dental powder composition of claim 10, wherein the anti-tartar control agent is selected from the group of sodium hexametaphosphate, phosphates and polyphosphates (for example pyrophosphates and tripolyphosphates), polyaminopropanesulfonic acid (AMPS), hexametaphosphate salts, zinc citrate trihydrate, polypeptides, polyolefin sulfonates, polyolefin phosphates, sodium acid phosphate (SAP), sodium acid pyrophosphate, and tripolyphosphate, and diphosphonates.

19. The dental powder composition of claim 10, wherein the anti-tartar control agent is in an amount from 0.1 to 5% by weight of the composition.

20. The dental powder composition of claim 10, wherein the botanical blend includes green tea and parsley, a therapeutic agent, a cosmetic agent, chlorophyll and sodium copper chlorophyllin, and/or a decorative/aesthetic agent.
